SEQUENCE LISTING (17 sequences)

SEQ ID NO: 1 - DNA, 40 bp, synthetic construct
  source 1..40: mol_type = other DNA
cctttgaccataggaggcaagtcttatgttcaaactggtc

SEQ ID NO: 2 - DNA, 40 bp, synthetic construct
  source 1..40: mol_type = other DNA
tcttgaaaacgccctgaagcggccttttcacgagggtacc

SEQ ID NO: 3 - DNA, 40 bp, synthetic construct
  source 1..40: mol_type = other DNA
gtagaacttggagagccaccttctaaggcgcagtcacacg

SEQ ID NO: 4 - DNA, 40 bp, synthetic construct
  source 1..40: mol_type = other DNA
ttgaacagaaccattgtccttcaatgcctagcaacctgtc

SEQ ID NO: 5 - DNA, 40 bp, synthetic construct
  source 1..40: mol_type = other DNA
gtataaacttagcagactttacacggcacacaaagctttc

SEQ ID NO: 6 - DNA, 40 bp, synthetic construct
  source 1..40: mol_type = other DNA
tggtgagtaaacataggtgcatactctacctttgctcatc

SEQ ID NO: 7 - DNA, 40 bp, synthetic construct
  source 1..40: mol_type = other DNA
ttgagagcggatgctagtttagttatagcctccgactctc

SEQ ID NO: 8 - DNA, 40 bp, synthetic construct
  source 1..40: mol_type = other DNA
atcataaatcatataacatgcaaaatctttatggtttatc

SEQ ID NO: 9 - DNA, 40 bp, synthetic construct
  source 1..40: mol_type = other DNA
ttaccttaacatactgcagtacttaagaggaggatggcag

SEQ ID NO: 10 - DNA, 41 bp, synthetic construct
  source 1..41: mol_type = other DNA
taaacgatcgaccctttaggtgcagcagcattatctgcgta

SEQ ID NO: 11 - DNA, 41 bp, synthetic construct
  source 1..41: mol_type = other DNA
gaacgggtaaacagctaatgtcgatttctctttgcccgctc

SEQ ID NO: 12 - RNA, 25 nt, synthetic construct
  source 1..25: mol_type = other RNA
  modified_base 16: mod_base = OTHER; note = Uracil
  modified_base 19: mod_base = OTHER; note = Uracil
gggagagcggaagcgtgctgggccc

SEQ ID NO: 13 - RNA, 27 nt, synthetic construct
  source 1..27: mol_type = other RNA
  modified_base 6: mod_base = OTHER; note = Uracil
  modified_base 10: mod_base = OTHER; note = Uracil
  modified_base 14: mod_base = OTHER; note = Uracil
  modified_base 25: mod_base = OTHER; note = Uracil
ccccctaggtagctggagacccaatac

SEQ ID NO: 14 - DNA, 72 bp, synthetic construct
  source 1..72: mol_type = other DNA
gggagagcggaagcgtgctgggcccctttgaccataggaggcaagtcttatgttcaaactggtccataaccc

SEQ ID NO: 15 - RNA, 32 nt, synthetic construct
  source 1..32: mol_type = other RNA
  modified_base 1: mod_base = OTHER; note = Uracil
  modified_base 2: mod_base = OTHER; note = Uracil
  modified_base 3: mod_base = OTHER; note = Uracil
  modified_base 9: mod_base = OTHER; note = Uracil
  modified_base 20: mod_base = OTHER; note = Uracil
  modified_base 22: mod_base = OTHER; note = Uracil
  modified_base 23: mod_base = OTHER; note = Uracil
  modified_base 25: mod_base = OTHER; note = Uracil
  modified_base 27: mod_base = OTHER; note = Uracil
  modified_base 28: mod_base = OTHER; note = Uracil
tttgaccataggaggcaagtcttatgttcaaa

SEQ ID NO: 16 - RNA, 26 nt, synthetic construct
  source 1..26: mol_type = other RNA
  modified_base 6: mod_base = OTHER; note = Uracil
  modified_base 17: mod_base = OTHER; note = Uracil
  modified_base 19: mod_base = OTHER; note = Uracil
  modified_base 20: mod_base = OTHER; note = Uracil
  modified_base 24: mod_base = OTHER; note = Uracil
  modified_base 25: mod_base = OTHER; note = Uracil
  modified_base 22: mod_base = OTHER; note = Uracil
gaccataggaggcaagtcttatgttc

SEQ ID NO: 17 - RNA, 20 nt, synthetic construct
  source 1..20: mol_type = other RNA
  modified_base 3: mod_base = OTHER; note = Uracil
  modified_base 14: mod_base = OTHER; note = Uracil
  modified_base 16: mod_base = OTHER; note = Uracil
  modified_base 17: mod_base = OTHER; note = Uracil
  modified_base 19: mod_base = OTHER; note = Uracil
cataggaggcaagtcttatg

(19) United States
(12) Patent Application Publication
(10) Pub. No.: US 2025/0207140 A1
(43) Pub. Date: Jun. 26, 2025

(54) RESPIRATORY TRACT INFECTION THERAPEUTICS AGAINST COVID-19

(71) Applicants: City of Hope, Duarte, CA (US); Apterna Limited, London (GB)

(72) Inventors: John J. Rossi, Duarte, CA (US); Min-Sun Song, Duarte, CA (US); Peter Bains, London (GB)

(21) Appl. No.: 18/846,414

(22) PCT Filed: Mar. 21, 2023
(86) PCT No.: PCT/US2023/064784

§ 371 Date: Sep. 12, 2024

Related U.S. Application Data
(60) Provisional application No. 63/322,197, filed on Mar. 21, 2022.

(51) Int. Cl. C12N 15/115; A61P 31/14

(52) U.S. Cl. CPC C12N 15/115; A61P 31/14; C12N 2310/16; C12N 2310/531

(57) ABSTRACT

Provided herein, inter alia, are compositions comprising nucleic acid compounds and methods of using the compositions for the prevention and treatment of respiratory diseases, including SARS-CoV-2 infections.

### CROSS-REFERENCE TO RELATED APPLICATIONS

This application claims the benefit of priority to U.S. Application No. 63/322,197 filed Mar. 21, 2022, the disclosure of which is incorporated by reference herein in its entirety.

### SEQUENCE LISTING

The material in the accompanying Sequence Listing is hereby incorporated by reference in its entirety. The accompanying file, named “048440-831001WO_SequenceListing.xml” was created on Mar. 21, 2023 and is 31,208 bytes, machine format IBM-PC, MS-Windows operating system.

### BACKGROUND

The emergence of severe acute respiratory syndrome coronavirus 2 (SARS-CoV-2) disease in China at the end of 2019 has caused an evolving global pandemic, which put the health and safety of the global community at a high risk.

The trimeric spike (S) protein of SARS-CoV-2 mediates viral entry and elicits an immune response. SARS-CoV-2 functional receptor-binding domain (RBD) is considered a major target for neutralizing antibodies upon infection and the focus of therapeutics and vaccine development. However, despite various antiviral agents and symptom-alleviating interventions under development, no effective drug treatment for COVID-19 has yet been identified. Therefore, there is a great demand for new therapeutics to advance the treatment of COVID-19. The present application provides solutions to these and other problems in the art.

### SUMMARY

Disclosed herein, inter alia, are nucleic acid compound-based drugs that can provide immediate neutralizing protection against SARS-CoV-2 infection by targeting the trimeric spike protein (S protein) of the virus.

Provided herein, inter alia, are novel inhaled nucleic acid molecules that comprise RNA aptamers that specifically bind to conserved and functional essential elements of SARS-CoV-2 S protein, as viral neutralizing agents, to prevent viral entry and infection. In embodiments, the nucleic acid molecules provided herein are able to specifically bind to the RBD subunit of the SARS-CoV-2 spike protein. In embodiments, the nucleic acid molecules provided herein are effective against SARS-CoV-2 original strain. In embodiments, the nucleic acid molecules provided herein are effective against SARS-CoV-2 mutant strains. In embodiments, an inhaled neutralizing RNA intervention provided herein has utility for both pre-exposure prophylaxis and immediate post-exposure treatment, and thus it may provide a first line of defense against SARS-CoV-2 and/or future SARS-CoV strains.

In aspects, provided herein is a nucleic acid molecule comprising a nucleic acid sequence, which is at least 80% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In aspects, provided herein is a nucleic acid molecule of SEQ ID NO: 14. In aspects, provided herein is a nucleic acid molecule of SEQ ID NO: 15. In aspects, provided herein is a nucleic acid molecule of SEQ ID NO: 16. In aspects, provided herein is a nucleic acid molecule of SEQ ID NO: 17.

In aspects, provided herein are pharmaceutical compositions comprising therapeutically effective amounts of the disclosed nucleic acid molecules and one or more additives or excipients. In aspects, the pharmaceutical compositions are administered by inhalation.

In aspects, provided herein are methods of treating or preventing a respiratory tract infection in a subject in need thereof, which comprise administering to the subject therapeutically effective amounts of the disclosed nucleic acid molecules

In embodiments, the nucleic acid compounds can also be formulated as cocktails to maximize neutralization potency and breadth. In embodiments, administration of the nucleic acid compounds by inhalation can further maximize delivery to the epithelial cilial cells of the upper and lower respiratory tract, the tissue sites of initial viral attachment and infection.

### BRIEF DESCRIPTION OF THE DRAWINGS

FIG. 1 is a schematic representation showing that SARS-CoV-2 enters into host cells by the interaction of the S protein with the ACE2 receptor.

FIG. 2 shows the scientific rationale for multimerization of neutralizing agents. (A) CryoEM structures of the trimeric SARS-CoV-2 S protein. Left panel: closed S trimer unsharpened cryo-EM map and two orthogonal views from the side and top of the atomic model of closed S trimer. Right panel: Partially open S timer unsharpened cryo-EM map (one SB domain is open), and two orthogonal views from the side and top of the atomic model of the closed S trimer. (B) Aptamer selection via in vitro SELEX. (C) Multivalent aptamers. (D) Neutralizing RNAs docking in the trimeric RBD. (E) Formulation of multivalent aptamers.

FIG. 3 is a schematic representation depicting RNA treatment in vivo in hACE2 mouse model. Two assays are designed: 1) nucleic acid compound as a pre-exposure intervention in which one dose of nucleic acid compound is applied to hACE2 mice at 4 h, 24 h or 48 h prior to SARS-CoV-2 infection; 2) nucleic acid compound as a post-exposure intervention in which four doses of nucleic acid compound are applied to SARS-CoV-2 infected hACE2 mice.

FIG. 4 shows the structure of a nucleic acid compound comprising SEQ ID NO: 1 bound to SEQ ID NO: 12 at its 5′end and to SEQ ID NO: 13 at its 3′ end (left) and the structure of a nucleic acid compound comprising SEQ ID NO: 2 bound to SEQ ID NO: 12 at its 5′end and to SEQ ID NO: 13 at its 3′ end (right).

FIG. 5 shows the structure of a nucleic acid compound comprising SEQ ID NO: 3 bound to SEQ ID NO: 12 at its 5′end and to SEQ ID NO: 13 at its 3′ end (left) and the structure of a nucleic acid compound comprising SEQ ID NO: 4 bound to SEQ ID NO: 12 at its 5′end and to SEQ ID NO: 13 at its 3′ end (right).

FIG. 6 shows the structure of a nucleic acid compound comprising SEQ ID NO: 5 bound to SEQ ID NO: 12 at its 5′end and to SEQ ID NO: 13 at its 3′ end.

FIG. 7 shows the structure of a nucleic acid compound comprising SEQ ID NO: 6 bound to SEQ ID NO: 12 at its 5′end and to SEQ ID NO: 13 at its 3′ end (left); the structure of a nucleic acid compound comprising SEQ ID NO: 7 bound to SEQ ID NO: 12 at its 5′end and to SEQ ID NO: 13 at its 3′ end (center); and the structure of a nucleic acid compound comprising SEQ ID NO: 8 bound to SEQ ID NO: 12 at its 5′end and to SEQ ID NO: 13 at its 3′ end (right).

FIG. 8 shows the structure of a nucleic acid compound comprising SEQ ID NO: 9 bound to SEQ ID NO: 12 at its 5′end and to SEQ ID NO: 13 at its 3′ end (left); the structure of a nucleic acid compound comprising SEQ ID NO: 10 bound to SEQ ID NO: 12 at its 5′end and to SEQ ID NO: 13 at its 3′ end (center); and the structure of a nucleic acid compound comprising SEQ ID NO: 11 bound to SEQ ID NO: 12 at its 5′end and to SEQ ID NO: 13 at its 3′ end (right).

FIG. 9 is a schematic representation of the in vitro SELEX protocol used to select 2′-fluoropyrimidine-modified RNA nucleic acid compounds that selectively bind to the S1 receptor-binding domain (RBD) of the recombinant Spike protein expressed in human embryonic kidney (HEK) 293 cells. The RBD was tagged with a six-histidine (His6) tag. An RNA aptamer library pool was first incubated with agarose beads to remove non-specific binders. The supernatant was then incubated with the His6-spike target protein for positive selection, and the aptamers bound to spike were amplified by PCR and in vitro transcription.

FIG. 10 shows the increase in the frequency of specific nucleic acid compound groups with each round of selection via SELEX. The molecular enrichment of each group of nucleic acid compounds is shown in the initial library (Lib), and after the 4th round and the 5th round of selection. The percent frequency of each group of nucleic acid compounds at each selection round was calculated by dividing the reads of each group by the total reads of the top 1000 unique sequences.

FIG. 11 shows the results of a neutralization assay with candidate nucleic acid compounds of SEQ ID NOS: 1-11, as measured by the InvivoGen™ cell fusion assay and detected by the SEAP detection reagent QUANTI-Blue™ Solution. Each bar corresponds to one of the candidate nucleic acid compounds of SEQ ID NOS: 1-11. The percentage of neutralization for each tested sample in cells expressing the original spike protein (A) or the omicron spike variant (B) was calculated based on the neutralization value for the original Library sample. The Library sample is a 40-nt randomized RNA sequence. Data represent the mean±standard deviation (SD). Student's t-test, non-parametric statistical tests and ANOVA (one-way and two-way) were performed for the statistical analysis. ns>0.05, *p<0.05, **p<0.01, ***p<0.001, ****p<0.0001.

FIG. 12 shows a 72 nucleotide-long truncated nucleic acid compound obtained from the nucleic acid compound of SEQ ID NO: 1.

FIG. 13 shows a 32 nucleotide-long truncated nucleic acid compound obtained from the nucleic acid compound of SEQ ID NO: 1.

FIG. 14 shows a 26 nucleotide-long truncated nucleic acid compound obtained from the nucleic acid compound of SEQ ID NO: 1.

FIG. 15 shows a 20 nucleotide-long truncated nucleic acid compound obtained from the nucleic acid compound of SEQ ID NO: 1.

FIG. 16 shows the ability of selected nucleic acid compounds to bind to the original Spike protein (A) or to the Spike Omicron variant (B) expressed in HEK293 cells, as compared to a nucleic acid compound (−) that targets CD8. Bars from left to right: SEQ ID NO: 1; truncated SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; control. The amount of nucleic acid compound bound to the cells was determined by flow cytometry and represented as percentage of input RNA.

FIG. 17 shows fluorescence binding curves for each nucleic acid compound as determined by titration of nucleic acid compound concentration. Binding curves of Cy3-labeled nucleic acid compounds on HEK293 cells expressing the original Spike protein (A) or the Omicron Spike variant (B) are shown. Binding was analyzed using flow cytometric assay. (C) Binding affinity (KD) values for the original spike protein as compared to the omicron variant Spike protein (D) expressed in HEK293 cells. The KD values are the mean: SD of 2-3 independent experiments.

### DETAILED DESCRIPTION

### Definitions

While various embodiments and aspects of the present invention are shown and described herein, it will be understood to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“Nucleic acid” refers to deoxyribonucleotides or ribonucleotides and polymers thereof in either single- or double-stranded forms, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The terms “transfection”, “transduction”, “transfecting” or “transducing” are used interchangeably throughout and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified (e.g., labeled).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region), when compared and aligned for maximum correspondence over a comparison window, or designated region as measured using one of the following sequence comparison algorithms or by manual alignment and visual inspection. Such sequences are then considered to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Any methods of alignment of sequences for comparison well known in the art are contemplated. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

Example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically or substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “aptamer” as provided herein refers to DNA or RNA oligonucleotides selected from random-sequence, single-stranded nucleic acid libraries by an in vitro selection and amplification procedure known as SELEX (systematic evolution of ligands by exponential enrichment) that bind with high affinity and specificity to proteins, peptides, and small molecules. Aptamers are capable of adapting unique tertiary structures and recognizing target molecules with high affinity and specificity. Binding affinities of aptamers are comparable to those of antibodies. Aptamers can be modified chemically at defined positions and linked stably to solid surfaces. Aptamers may have secondary or tertiary structure and, thus, may be able to fold into diverse and intricate molecular structures. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12): e15004). Applying the SELEX and the SOMAmer technology includes for instance adding functional groups that mimic amino acid side chains to expand the aptamer's chemical diversity. As a result high affinity aptamers for a protein may be enriched and identified. Aptamers may exhibit many desirable properties for targeted drug delivery, such as ease of selection and synthesis, high binding affinity and specificity, low immunogenicity, and versatile synthetic accessibility. Aptamers may be DNA or RNA molecules and may be single stranded or double stranded. The aptamer may comprise chemically modified nucleotides or nucleosides, for example in which the sugar and/or phosphate and/or base is chemically modified. Such modifications may improve the stability of the aptamer or make the aptamer more resistant to degradation. The aptamers provided herein may include chemical modifications as described herein such as a chemical substitution at a sugar position, a phosphate position, and/or a base position of the nucleic acid including, for example, incorporation of a modified nucleotide, incorporation of a capping moiety (e.g. 3′ capping), conjugation to a high molecular weight, non-immunogenic compound (e.g. polyethylene glycol (PEG)), conjugation to a lipophilic compound, substitutions in the phosphate backbone. Base modifications may include 5-position pyrimidine modifications, modifications at exocyclic amines, substitution of 4-thiouridine, substitution of 5-bromo- or 5-iodo-uracil, backbone modifications. Sugar modifications may include 2′-amine nucleotides (2′-NH2), 2′-fluoro nucleotides (2′-F), and 2′-O-methyl (2′-OMe) nucleotides. In embodiments, aptamers are modified with 2′-fluoro in U and C nucleotides. In embodiments, the aptamers described herein may comprise one or more modified nucleotides. Exemplary modifications include, but are not limited to, nucleotides comprising an alkylation, arylation, acetylation, alkoxylation, halogenation, amino groups, or other functional groups. Examples of modified nucleotides include 2′-fluoro ribonucleotides, 2′-NH—, 2′-OCH3-, and 2′-0-methoxyethyl ribonucleotides. A wide range of nucleotide, nucleoside, base and phosphate modifications are known to those or ordinary skill in the art, e.g. as described in Eaton et al., Bioorganic 8z Medicinal Chemistry, Vol. 5, No. 6, pp 1087-1096, 1997. Aptamers may be synthesised by methods which are well known to the skilled person. For example, aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D.; Biemat, J.; McManus, J.; KOster, H. Nucleic Acids Res. 1984, 12, 4539; and Beaucage, S. L.; Lyer, R. P. (1992). Tetrahedron 48 (12): 2223). Aptamer quantities are often in the nM or pM range, e.g. less than one of 500 nM, 100 nM, 50 nM, 10 nM, 1 nM, 500 pM, 100 pM. Aptamers have use in therapeutic and diagnostic applications, in vitro or in vivo. In vitro diagnostic applications may include use in detecting the presence or absence of a target molecule. Aptamers described herein may be provided in purified or isolated form. Aptamers described herein may be formulated as a pharmaceutical composition or medicament. In embodiments, the aptamers provided herein are able to specifically bind to the RBD subunit of the SARS-CoV-2 spike protein. In embodiments, the nucleic acid sequence of an aptamer may optionally have a minimum length of one of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In embodiments, the nucleic acid sequence of an aptamer, may optionally have a maximum length of one of 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In embodiments, the nucleic acid sequence of an aptamer, may optionally have a length of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In embodiments, the nucleic acid sequence of an aptamer, may have a degree of primary sequence identity with one of SEQ ID NOs 1 to 11, that is at least one of 80%, 81%, 82%, 83%, 84%, 85%, 86%, 87%, 88%, 89%, 90%, 91%, 92%, 93%, 94%, 95%, 96%, 97%, 98%, 99%, or 100%. In embodiments, the aptamers provided here are RNA aptamers.

An “antisense nucleic acid” as referred to herein is a nucleic acid (e.g. DNA or RNA molecule) that is complementary to at least a portion of a specific target nucleic acid (e.g. an mRNA translatable into a protein) and is typically capable of reducing transcription of the target nucleic acid (e.g. mRNA from DNA) or reducing the translation of the target nucleic acid (e.g. mRNA) or altering transcript splicing (e.g. single stranded morpholino oligo). See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone modified nucleotides. In the cell, the antisense nucleic acids may hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (Marcus-Sakura, Anal. Biochem. 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogues), short hairpin RNAs (shRNA), micro RNAs (miRNA), saRNAs (small activating RNAs) and small nucleolar RNAs (snoRNA) or certain of their derivatives or pre-cursors.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when present in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

A “saRNA,” or “small activating RNA” as provided herein refers to a nucleic acid that forms a double stranded RNA, which double stranded RNA has the ability to increase or activate expression of a gene or target gene when present in the same cell as the gene or target gene. The complementary portions of the nucleic acid that hybridize to form the double stranded molecule typically have substantial or complete identity. In one embodiment, a saRNA is a nucleic acid that has substantial or complete identity to a target gene and forms a double stranded saRNA. Typically, the nucleic acid is at least about 15-50 nucleotides in length (e.g., each complementary sequence of the double stranded saRNA is 15-50 nucleotides in length, and the double stranded saRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

The ribonucleic acid compounds described herein can be co-administered with or be covalently attached to conventional immunotherapeutic agents including, but not limited to, immunostimulants (e.g., Bacillus Calmette-Guerin (BCG), levamisole, interleukin-2, alphainterferon, etc.), monoclonal antibodies (e.g., anti-CD20, anti-HER2, anti-CD52, anti-HLA-DR, anti-PD-1 and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to 111In, ⁹⁰Y, or ¹³¹I, etc.).

The ribonucleic acid compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, 901., 105Rh, 111A_{g}, 1111ₙ, 117sₙ, 149Pm, 153Sm, ¹⁶⁶HO, ¹⁷⁷LU, 186Re, 188Rₑ, 211At, and ²¹¹Bi, optionally conjugated to antibodies directed against antigens.

The term “nucleotide” typically refers to a compound containing a nucleoside or a nucleoside analogue and at least one phosphate group or a modified phosphate group linked to it by a covalent bond. Exemplary covalent bonds include, without limitation, an ester bond between the 3′, 2′ or 5′ hydroxyl group of a nucleoside and a phosphate group.

The term “nucleoside” refers to a compound containing a sugar part and a nucleobase, e.g., a pyrimidine or purine base. Exemplary sugars include, without limitation, ribose, 2-deoxyribose, arabinose and the like. Exemplary nucleobases include, without limitation, thymine, uracil, cytosine, adenine, guanine.

The term “nucleoside analogue” may refer to a nucleoside any part of which is replaced by a chemical group of any nature. Exemplary nucleoside analogues include, without limitation, 2′-substituted nucleosides such as 2′-fluoro, 2-deoxy, 2′-O-methyl, 2′-O—P-methoxyethyl, 2′-O-allylriboribonucleosides, 2′-amino, locked nucleic acid (LNA) monomers and the like. The term “nucleoside analogue” may also refer to a nucleoside in which the sugar or base part is modified, e.g. with a non-naturally occurring modification. Exemplary nucleoside analogues in which the sugar part is replaced with another cyclic structure include, without limitation, monomeric units of morpholinos (PMO) and tricyclo-DNA. Exemplary nucleoside analogues in which the sugar part is replaced with an acyclic structure include, without limitation, monomeric units of peptide nucleic acids (PNA) and glycerol nucleic acids (GNA). Suitably, nucleoside analogues may include nucleoside analogues in which the sugar part is replaced by a morpholine ring.

In structures of this type, it will be appreciated that the labels 3′ and 5′, as applied to conventional sugar chemistry, apply by analogy. That is, in the structure depicted, the hydroxylmethyl substituent on the ring is considered the 5′ end, while the third nitrogen valency is considered the 3′ end.

Nucleoside analogues may include deoxyadenosine analogues, adenosine analogues, deoxycytidine analogues, cytidine analogues, deoxyguanosine analogues, guanosine analogues, thymidine analogues, 5-methyluridine analogues, deoxyuridine analogues, or uridine analogues. Examples of deoxyadenosine analogues include didanosine (2′, 3′-dideoxyinosine) and vidarabine (9-O-D-arabinofuranosyladenine), fludarabine, pentostatin, cladribine. Examples of adenosine analogues include DCX4430 (Immucillin-A). Examples of cytidine analogues include gemcitabine, 5-aza-2′-deoxycytidine, cytarabine. Examples of deoxycytidine analogues include cytarabine, emtricitabine, lamivudine, zalcitabine. Examples of guanosine and deoxyguanosine analogues include abacavir, acyclovir, entecavir. Examples of thymidine and 5-methyluridine analogues include stavudine, telbivudine, zidovudine. Examples of deoxyuridine analogues include idoxuridine and trifluridine.

The terms “purine analogue” or “pyrimidine analogue” refers to modifications, optionally non-naturally occurring modifications, in the nucleobase, for example hypoxanthine, xanthine, 2-aminopurine, 2,6-diaminopurine, 6-azauracil, 5-methylcytosine, 4-fluorouracil, 5-fluoruracil, 5-chlorouracil, 5-bromouracil, 5-iodouracil, 5-trifluoromethyluracil, 5-fluorocytosine, 5-chlorocytosine, 5-bromocytosine, 5-iodocytosine, 5-propynyluracil, 5-propynylcytosine, 7-deazaadenine, 7-deazaguanine, 7-deaza-8-azaadenine, 7-deaza-8-azaguanine, isocytosine, isoguanine, mercaptopurine, thioguanine. Exemplary pyrimidine analogues include, without limitation, 5-position substituted pyrimidines, e.g. substitution with 5-halo, 5′-fluoro. Examples of purine analogues include, without limitation, 6- or 8-position substituted purines, e.g., substitution with 5-halo, 5′-fluoro.

The term “phosphate group” as used herein refers to phosphoric acid H3PO4 wherein any hydrogen atoms are replaced by one, two or three organic radicals to give a phosphoester, phosphodiester, or phosphotriester, respectively. Oligonucleotides may be linked by phosphodiester, phosphorothioate or phosphorodithioate linkages.

In aptamers described herein, one or more or each adenine, adenosine or deoxyadenosine in the aptamer oligonucleotide sequence may be replaced, e.g. substituted, with an adenine, adenosine or deoxyadenosine analogue. Similarly, in aptamers described herein, one or more or each cytosine, cytidine or deoxycytidine in the aptamer oligonucleotide sequence may be replaced, e.g. substituted, with a cytosine analogue, cytidine analogue or deoxycytidine analogue. Additionally, in aptamers described herein, one or more or each guanine, guanosine or deoxyguanosine in the aptamer oligonucleotide sequence may be replaced, e.g. substituted, with a guanine, guanosine or deoxyguanosine analogue. Furthermore, in aptamers described herein, one or more or each thymine, 5-methyluridine or thymidine in the aptamer oligonucleotide sequence may be replaced, e.g. substituted, with a thymine, 5-methyluridine or thymidine analogue. In aptamers described herein, one or more or each uracil, uridine or deoxyuridine in the aptamer oligonucleotide sequence may be replaced, e.g. substituted, with a uracil, uridine or deoxyuridine analogue.

By the term “SARS-CoV-2” it is meant the virus that causes a respiratory disease known as coronavirus disease 19 (COVID-19) or severe acute respiratory syndrome coronavirus 2. SARS-CoV-2 is a member of a large family of coronaviruses. These viruses can infect people and some animals. SARS-CoV-2 was first known to infect people in 2019. The virus is thought to spread from person to person through droplets released when an infected person coughs, sneezes, or talks. It may also be spread by touching a surface with the virus on it and then touching one's mouth, nose, or eyes, but this is less common. Research is being done to treat COVID-19 and to prevent infection with SARS-CoV-2.

The term “Spike (S) glycoprotein” or “spike protein” is used in accordance with its plan and ordinary meaning and, for example, refers to the largest of the four major structural proteins found in coronaviruses. The spike protein assembles into trimers that form large structures, called spikes or peplomers, that project from the surface of the virion. The function of the spike glycoprotein is to mediate viral entry into the host cell by first interacting with molecules on the exterior cell surface and then fusing the viral and cellular membranes. Spike glycoprotein is a class I fusion protein that contains two regions, known as S1 and S2, responsible for these two functions. The S1 region contains the receptor-binding domain that binds to receptors on the cell surface. Coronaviruses use a very diverse range of receptors; SARS-CoV (which causes SARS) and SARS-CoV-2 (which causes COVID-19) both interact with angiotensin-converting enzyme 2 (ACE2). The S2 region contains the fusion peptide and other fusion infrastructure necessary for membrane fusion with the host cell, a required step for infection and viral replication.

The term “sample” includes sections of tissues such as biopsy and autopsy samples, and frozen sections taken for histological purposes. Such samples include blood and blood fractions or products (e.g., bone marrow, serum, plasma, platelets, red blood cells, and the like), sputum, tissue, cultured cells (e.g., primary cultures, explants, and transformed cells), stool, urine, other biological fluids (e.g., prostatic fluid, gastric fluid, intestinal fluid, renal fluid, lung fluid, cerebrospinal fluid, and the like), etc. A sample is typically obtained from a “subject” such as a eukaryotic organism, most preferably a mammal such as a primate, e.g., chimpanzee or human; cow; dog; cat; a rodent, e.g., guinea pig, rat, mouse; rabbit; or a bird; reptile; or fish.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms; fully or partially remove the disease's underlying cause; shorten a disease's duration; or do a combination of these things.

“Treating” and “treatment” as used herein also include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington. The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain and ordinary meaning and includes oral administration, administration by inhalation, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

By “Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Selective” or “selectivity” or the like of a compound refers to the compound's ability to discriminate between molecular targets.

“Specific”, “specifically”, “specificity”, or the like of a compound refers to the compound's ability to cause a particular action, such as inhibition, to a particular molecular target with minimal or no action to other proteins in the cell.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The term “expression” includes any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “associated” or “associated with” in the context of a substance or substance activity or function associated with a disease (e.g., a protein associated with an infectious disease) means that the disease (is caused by (in whole or in part), or a symptom of the disease is caused by (in whole or in part) the substance or substance activity or function. As used herein, what is described as being associated with a disease, if a causative agent, could be a target for treatment of the disease. For example, a symptom associated with a disease, such as COVID-19 may be treated with an S protein modulator or S protein inhibitor, in the instance where S protein activity or function causes the disease (e.g., COVID-19).

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

“Pharmaceutically acceptable excipient” and “pharmaceutically acceptable carrier” refer to a substance that aids the administration of an active agent to and absorption by a subject and can be included in the compositions described herein without causing a significant adverse toxicological effects on the subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylase or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions described herein. One of skill in the art will recognize that additional pharmaceutical excipients may be useful. The term “pharmaceutically acceptable salt” refers to salts derived from a variety of organic and inorganic counter ions well known in the art and include, by way of example only, sodium, potassium, calcium, magnesium, ammonium, tetraalkylammonium, and the like.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g. a first linker or second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der Waal's bond(s)/interactions, hydrogen bond(s), polar bond(s), or combinations or mixtures thereof).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Nucleic Acid Compounds

Provided herein, inter alia, are nucleic acid compounds comprising aptamers that are apable of specifically binding SARS-CoV-2 S protein RBD domains, as viral neutralizing agents, to prevent viral entry and infection. The trimeric spike (S) protein of SARS-CoV-2 mediates viral entry via its receptor-binding domain (RBD). In embodiments, the nucleic acid compounds provided herein block the initial viral entry, thereby preventing infection. In embodiments, the nucleic acid compounds provided herein can be easily administered by inhalation, thus providing an efficient mechanism for targeted intracellular delivery.

In embodiments, the disclosed nucleic acid compounds contain RNA aptamers that target and bind SARS-CoV-2 S protein RBD domains and the ACE2 receptor with high affinity and specificity. Aptamers may present several advantages compared to traditional approaches. In embodiments, aptamers are capable of tissue penetration and differential accessibility to highly conserved binding sites between RBD and ACE2. In embodiments, aptamers are amenable to molecular engineering approaches, including generation of oligomers, conjugates and other multi-functional derivatives. In embodiments, RNAs are adaptable to formulation for inhaled use. Inhalation administration delivers the drug directly to the respiratory tract, which may be the primary site of viral entry.

In embodiments, provided herein is a nucleic acid molecule that comprises a nucleic acid sequence, which is at least 80% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In embodiments, the nucleic acid sequence has at least 85%, at least 87%, at least 90%, at least 91%, at least 92%, at least 93%, at least 94%, at least 95%, at least 96%, at least 97%, at least 98% or at least 99% sequence identity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In embodiments, the nucleic acid sequence has at least 90% sequence identity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In embodiments, the sequence has 80%, 85%, 87%, 90%, 91%, 92%, 93%, 94%, 95%, 96%, 97%, 98%, 99% or 100% sequence identity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In embodiments, the sequence has 100% sequence identity to SEQ ID NO:1, SEQ ID NO: 2, SEQ ID NO: 3, SEQ ID NO: 4, SEQ ID NO: 5. SEQ ID NO: 6, SEQ ID NO: 7, SEQ ID NO: 8, SEQ ID NO: 9, SEQ ID NO: 10, or SEQ ID NO: 11.

In embodiments, provided herein is a nucleic acid molecule that comprises SEQ ID NO: 14. In embodiments, the nucleic acid molecule does not comprise any portion of SEQ ID NO: 1 other than SEQ ID NO: 14.

In embodiments, provided herein is a nucleic acid molecule that comprises SEQ ID NO: 15. In embodiments, the nucleic acid molecule does not comprise any portion of SEQ ID NO: 1 other than SEQ ID NO: 15.

In embodiments, provided herein is a nucleic acid molecule that comprises SEQ ID NO: 16. In embodiments, the nucleic acid molecule does not comprise any portion of SEQ ID NO: 1 other than SEQ ID NO: 16.

In embodiments, provided herein is a nucleic acid molecule that comprises SEQ ID NO: 17. In embodiments, the nucleic acid molecule does not comprise any portion of SEQ ID NO: 1 other than SEQ ID NO: 17.

In embodiments, provided herein is a nucleic acid molecule of SEQ ID NO: 14.

In embodiments, provided herein is a nucleic acid molecule of SEQ ID NO: 15.

In embodiments, provided herein is a nucleic acid molecule of SEQ ID NO: 16.

In embodiments, provided herein is a nucleic acid molecule of SEQ ID NO: 17.

In embodiments, the sequence is an RNA sequence that is capable of binding to a receptor binding domain of the SARS-CoV-2 S protein.

In embodiments, the nucleic acid molecules as described herein may further comprise additional nucleotide sequences. Additional nucleotide sequences may be added onto the 5′ end, the 3′ end, or both the 5′ and 3′ ends of the RNA sequence. In embodiments, an RNA sequence comprising SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 as described herein comprises a total of 1, 2, 3, 4, 5, 6, 7, 8, 9, 10, 11, 12, 13, 14, 15, 16, 17, 18, 19, 20, 21, 22, 23, 24, 25, 26, 27, or more additional nucleotides.

In embodiments, a nucleic acid molecule described herein, such as SEQ ID NO: 14, SEQ ID NO: 15, SEQ ID NO: 16, and SEQ ID NO: 17, comprise a total of 72, 32, 26, 20, or less nucleotides.

In embodiments, the RNA sequence may be attached to a nucleic acid sequence at its 5′ end. In embodiments, the RNA sequence may be attached to a nucleic acid sequence at its 3′ end. In embodiments, the RNA sequence may be attached to a first nucleic acid sequence at its 5′ end and to a second nucleic acid sequence at its 3′ end.

In embodiments, each of the nucleotide sequence attached to the 5′ end of the RNA sequence and the nucleotide sequence attached to the 3′ end of the RNA sequence comprises at least one stem loop structure. In embodiments, the nucleotide sequence attached to the 5′ end of the RNA sequence or the nucleotide sequence attached to the 3′ end of the RNA sequence comprises at least one stem loop structure. In embodiments, the nucleotide sequence attached to the 5′ end of the RNA sequence and the nucleotide sequence attached to the 3′ end of the RNA sequence are combined to form at least one stem loop structure.

In embodiments, the nucleic acid molecules disclosed herein may comprise one or more stem loop structures comprising portions of the RNA sequence, portions of the nucleotide sequence attached to the 5′ end of the RNA sequence, and portions of the nucleotide sequence attached to the 3′ end of the RNA sequence. In embodiments, the RNA sequence may also comprise one or more stem loop structures.

Without being bound to any theory, it is postulated that stem loop structures increase the stability of the nucleic acid molecules provided herein and enhance their target binding affinity.

In embodiments, the nucleotide sequence attached to the 5′ end of the RNA sequence may comprise a sequence which is at least 80% identical to SEQ ID NO: 12. In embodiments, the nucleotide sequence attached to the 5′ end of the RNA sequence may comprise a sequence which is at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12.

In embodiments, the nucleotide sequence attached to the 3′ end of the RNA sequence may comprise a sequence which is at least 80% identical to SEQ ID NO: 13. In embodiments, the nucleotide sequence attached to the 3′ end of the RNA sequence may comprise a sequence which is at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the RNA sequence is attached to a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 at its 5′ end. In embodiments, the RNA sequence is attached to a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 at its 3′ end. In embodiments, the RNA sequence is attached to a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 at its 5′ end, and to a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 at its 3′ end.

In embodiments, the nucleic acid molecule comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:1, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:1, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:2, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:2, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:3, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:3, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:4, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO: 4, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:5, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO: 5, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:6, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO: 6, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:7, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:7, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:8, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:8, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:9, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:9, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:10, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:10, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:11, and further comprises a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 attached to the 5′ end of the RNA sequence, and a nucleic acid sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13 attached to the 3′ end of the RNA sequence.

In embodiments, the nucleic acid compound comprises a stem loop structure that comprises a portion of an RNA sequence which is at least 80%, at least 85%, at least 87%, at least 90%, at least 91%, at least 92%, at least 93%, at least 94%, at least 95%, at least 96%, at least 97%, at least 98% or at least 99% sequence identity to SEQ ID NO:11, a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12 and a portion of a nucleotide sequence which is at least 80%, at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

In embodiments, the nucleic acid compound comprises a sequence that is at least 80%, at least 85%, at least 87%, at least 90%, at least 91%, at least 92%, at least 93%, at least 94%, at least 95%, at least 96%, at least 97%, at least 98% or at least 99% identical to SEQ ID NO:14, and does not comprise any other portion of SEQ ID NO: 1.

In embodiments, the nucleic acid compound comprises a sequence that is at least 80%, at least 85%, at least 87%, at least 90%, at least 91%, at least 92%, at least 93%, at least 94%, at least 95%, at least 96%, at least 97%, at least 98% or at least 99% identical to SEQ ID NO:15, and does not comprise any other portion of SEQ ID NO: 1.

In embodiments, the nucleic acid compound comprises a sequence that is at least 80%, at least 85%, at least 87%, at least 90%, at least 91%, at least 92%, at least 93%, at least 94%, at least 95%, at least 96%, at least 97%, at least 98% or at least 99% identical to SEQ ID NO:16, and does not comprise any other portion of SEQ ID NO: 1.

In embodiments, the nucleic acid compound comprises a sequence that is at least 80%, at least 85%, at least 87%, at least 90%, at least 91%, at least 92%, at least 93%, at least 94%, at least 95%, at least 96%, at least 97%, at least 98% or at least 99% identical to SEQ ID NO:17, and does not comprise any other portion of SEQ ID NO: 1.

In embodiments, the nucleic acid compound is at least 80%, at least 85%, at least 87%, at least 90%, at least 91%, at least 92%, at least 93%, at least 94%, at least 95%, at least 96%, at least 97%, at least 98% or at least 99% identical to SEQ ID NO: 14.

In embodiments, the nucleic acid compound is at least 80%, at least 85%, at least 87%, at least 90%, at least 91%, at least 92%, at least 93%, at least 94%, at least 95%, at least 96%, at least 97%, at least 98% or at least 99% identical to SEQ ID NO: 15.

In embodiments, the nucleic acid compound is at least 80%, at least 85%, at least 87%, at least 90%, at least 91%, at least 92%, at least 93%, at least 94%, at least 95%, at least 96%, at least 97%, at least 98% or at least 99% identical to SEQ ID NO: 16.

In embodiments, the nucleic acid compound is at least 80%, at least 85%, at least 87%, at least 90%, at least 91%, at least 92%, at least 93%, at least 94%, at least 95%, at least 96%, at least 97%, at least 98% or at least 99% identical to SEQ ID NO: 17.

In embodiments, the disclosed nucleic acid compounds are RNase-resistant.

### Pharmaceutical Compositions

In further embodiments, provided herein are pharmaceutical compositions comprising anyone of the nucleic acid compounds in therapeutically effective amounts and one or more pharmaceutically acceptable additives or excipients.

In embodiments, the pharmaceutical compositions are in form of aerosol, nasal spray, inhalers, or mouth spray and are administered by inhalation. In embodiments, the pharmaceutical compositions provided herein are administered intratumorally, intranodally, transdermally, intravenously, and through lymphatic transport.

In embodiments, the pharmaceutical compositions may further comprise a therapeutic agent such as, but not limited to, a vaccine, an anti-inflammatory agent, an antibody, a short-acting beta agonist, an antipyretic agent or an immune booster.

For any of the compositions comprising the nucleic acid compounds described herein, the therapeutically effective amount can be initially determined from cell culture assays. Target concentrations will be those concentrations of active compound(s) that are capable of achieving the methods described herein, as measured using the methods described herein or known in the art. Effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

The provided compositions are, inter alia, suitable for formulation and administration in vitro or in vivo. Suitable carriers and excipients and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

Pharmaceutical compositions provided herein include compositions wherein the active ingredient (e.g., the nucleic acid compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, the recombinant proteins described herein will contain an amount of active ingredient effective to achieve the desired result, e.g., modulating the activity of a target molecule, and/or reducing, eliminating, or slowing the progression of disease symptoms. Determination of a therapeutically effective amount of a compound provided herein s well within the capabilities of those skilled in the art, especially in light of the detailed disclosure herein.

The pharmaceutical compositions provided herein may include a single active agent or more than one active agent. In embodiments, the pharmaceutical compositions further include a nucleic acid compound as described herein and an additional active agent. Thus, provided herein are compositions including pa nucleic acid compound as described herein and one or more therapeutic agents. Exemplary therapeutic agents include, but are not limited to, vaccines, anti-inflammatory agents, antibodies, short-acting beta agonists, antipyretic agents, and immune boosters.

The pharmaceutical compositions for administration will commonly include an agent as described herein dissolved in a pharmaceutically acceptable carrier, preferably an aqueous carrier. A variety of aqueous caters can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the subject's needs.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

The pharmaceutical compositions provided herein can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

The pharmaceutical compositions provided herein can also be combined with pharmaceutically acceptable carriers or additives and delivered intratumorally, intranodally, intravenously, or through lymphatic transport.

The formulations of compounds can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for inhalation include, but are not limited to, aerosols, inhalers, and the like.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of SARS-Co-V2 and severity of such symptoms), kind of concurrent treatment, complications from the disease being treated or other health-related problems. Other therapeutic regimens or agents can be used in conjunction with the methods and compounds provided herein. Adjustment and manipulation of established dosages (e.g., frequency and duration) are well within the ability of those skilled in the art.

For any composition of the nucleic acid compounds provided herein, the therapeutically effective amount can be initially determined from cell culture assays. Target concentrations will be those concentrations of active compound(s) that are capable of achieving the methods described herein, as measured using the methods described herein or known in the art. As is well known in the art, effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred

“Pharmaceutically acceptable excipient” and “pharmaceutically acceptable carrier” refer to a substance that aids the administration of an active agent to and absorption by a subject and can be included in the compositions provided herein without causing a significant adverse toxicological effect on the patient. Non limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds provided herein. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

### Methods of Prevention and Treatment

Provided herein are methods of treating or preventing a respiratory tract infection in a subject in need thereof wherein the disclosed methods comprise administering to the subject a therapeutically effective amount of the nucleic acid compounds described herein. In embodiments, therapeutically effective amounts of the nucleic acid compounds described herein are administered by inhalation in form of aerosol, nasal spray, inhalers or mouth spray. In embodiments, therapeutically effective amount of the nucleic acid compounds described herein are administered once or twice a day.

Additionally provided herein are methods of treating or preventing a respiratory tract infection in a subject in need thereof wherein the disclosed methods comprise administering to the subject the pharmaceutical compositions described herein. In embodiments, the pharmaceutical compositions are administered by inhalation in form of aerosol, nasal spray, inhalers or mouth spray. In embodiments, the pharmaceutical compositions provided herein are administered intratumorally, intranodally, transdermally, intravenously, and through lymphatic transport. In embodiments, the pharmaceutical compositions are administered once or twice a day.

The respiratory tract infection can be in the upper respiratory tract or in the lower respiratory tract of the subject. In embodiments, the respiratory tract infection is a SARS infection. In some aspects, the respiratory tract infection is a SARS-CoV-2 infection. In embodiments, the subject suffers from acute respiratory distress symptoms.

The methods provided herein may further comprise administering to the subject one or more therapeutic agents for treating or preventing one or more respiratory diseases such as, but not limited to, asthma, COPD, pneumonia, sinusitis, laryngitis, bronchitis, a chest infection, or a cold. In embodiments, the pharmaceutical compositions and the one or more therapeutic agents are administered separately. In embodiments, the pharmaceutical compositions and the one or more therapeutic agents are administered together.

For prophylactic benefit, the pharmaceutical compositions comprising the nucleic acid compounds described herein may be administered to a subject at risk of developing a particular disease, such as a respiratory tract infection, or to a subject reporting one or more of the physiological symptoms of a disease, such as COVID-19, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The methods of treatment provided herein may further include administration of additional therapeutic agents that are suitable to the disease being treated. Thus, in some embodiments, the provided methods of treatment further comprise administering a second therapeutic agent to the subject. Suitable additional therapeutic agents include, but are not limited to, analgesics, anesthetics, analeptics, corticosteroids, anticholinergic agents, anticonvulsants, antirheumatic agents, anti-inflammatory agents, antibiotics, anticoagulants, antifungals, antihistamines, antimuscarinic agents, antimycobacterial agents, antiprotozoal agents, antiviral agents, immunological agents, vitamins, growth factors, and hormones. The choice of agent and dosage can be determined readily by one of skill in the art based on the given disease being treated.

Combinations of agents or compositions can be administered either concomitantly (e.g., as a mixture), separately but simultaneously (e.g., via separate administration) or sequentially (e.g., one agent is administered first followed by administration of the second agent). Thus, the term combination is used to refer to concomitant, simultaneous or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

According to the methods provided herein, the subject is administered an effective amount of one or more of the agents provided herein. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

The terms “subject,” “patient,” “individual,” etc. are not intended to be limiting and can be generally interchanged. That is, an individual described as a “patient” does not necessarily have a given disease, but may be merely seeking medical advice.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The compositions provided herein may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

### EMBODIMENTS

Embodiment 1. A nucleic acid molecule comprising a nucleic acid sequence, which is at least 80% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

Embodiment 2. The nucleic acid molecule of Embodiment 1, wherein the nucleic acid sequence is at least 85%, at least 90%, at least 95%, or at least 99% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

Embodiment 3. The nucleic acid molecule of Embodiment 1 or Embodiment 2, wherein the nucleic acid sequence is 100% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

Embodiment 4. The nucleic acid molecule of any one of Embodiments 1-3, wherein the nucleic acid sequence is an RNA sequence.

Embodiment 5. The nucleic acid molecule of Embodiment 4, wherein the nucleic acid molecule further comprises a nucleotide sequence, and wherein the RNA sequence is attached at its 5′ end to the nucleotide sequence.

Embodiment 6. The nucleic acid molecule of Embodiment 4, wherein the nucleic acid molecule further comprises a nucleotide sequence, and wherein the RNA sequence is attached at its 3′ end to the nucleotide sequence.

Embodiment 7. The nucleic acid molecule of Embodiment 4, wherein the nucleic acid molecule further comprises a first nucleotide sequence and a second nucleotide sequence, and wherein the RNA sequence is attached at its 5′ end to the first nucleotide sequence and at its 3′ end to the second nucleotide sequence.

Embodiment 8. The nucleic acid molecule of any one of Embodiments 6-7, wherein (a) each nucleotide sequence attached to the 5′ end of the RNA sequence and the nucleotide sequence attached to the 3′end of the RNA sequence comprises at least one stem loop structure; or (b) the nucleotide sequence attached to the 5′ end of the RNA sequence or the nucleotide sequence attached to the 3′ end of the RNA sequence comprises at least one stem loop structure; or (c) the nucleotide sequence attached to the 5′ end of the RNA sequence and the nucleotide sequence attached to the 3′ end of the RNA sequence are combined to form at least one stem loop structure.

Embodiment 9. The nucleic acid molecule of Embodiment 8, wherein the nucleic acid molecule comprises a stem loop structure comprising a portion of the RNA sequence, a portion of the nucleotide sequence attached to the 5′ end of the RNA sequence, and a portion of the nucleotide sequence attached to the 3′ end of the RNA sequence

Embodiment 10. The nucleic acid molecule of Embodiment 9, wherein the RNA sequence comprises one or more stem loop structures.

Embodiment 11. The nucleic acid molecule of any one of Embodiments 5 and 7-10, wherein the nucleotide sequence attached to the 5′ end of the RNA sequence comprises a sequence which is at least 80% identical to SEQ ID NO: 12.

Embodiment 12. The nucleic acid molecule of Embodiment 11, wherein the nucleotide sequence attached to the 5′ end of the RNA sequence comprises a sequence which is at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 12.

Embodiment 13. The nucleic acid molecule of any one of Embodiments 6-10, wherein the nucleotide sequence attached to the 3′ end of the RNA sequence comprises a sequence which is at least 80% identical to SEQ ID NO: 13.

Embodiment 14. The nucleic acid molecule of Embodiment 13, wherein the nucleotide sequence attached to the 3′ end of the RNA sequence comprises a sequence which is at least 85%, at least 87%, at least 90%, at least 91%, at least 92%, at least 93%, at least 94%, at least 95%, at least 96%, at least 97%, at least 98%, at least 99%, or at least 100% identical to SEQ ID NO: 13.

Embodiment 15. A nucleic acid molecule comprising SEQ ID NO: 14, wherein the nucleic acid molecule does not comprise any portion of SEQ ID NO: 1 other than SEQ ID NO: 14.

Embodiment 16. A nucleic acid molecule comprising SEQ ID NO: 15, wherein the nucleic acid molecule does not comprise any portion of SEQ ID NO: 1 other than SEQ ID NO: 15.

Embodiment 17. A nucleic acid molecule comprising SEQ ID NO: 16, wherein the nucleic acid molecule does not comprise any portion of SEQ ID NO: 1 other than SEQ ID NO: 16.

Embodiment 18. A nucleic acid molecule comprising SEQ ID NO: 17, wherein the nucleic acid molecule does not comprise any portion of SEQ ID NO: 1 other than SEQ ID NO: 17.

Embodiment 19. A nucleic acid molecule of SEQ ID NO: 14.

Embodiment 20. A nucleic acid molecule of SEQ ID NO: 15.

Embodiment 21. A nucleic acid molecule of SEQ ID NO: 16.

Embodiment 22. A nucleic acid molecule of SEQ ID NO: 17.

Embodiment 23. The nucleic acid molecule of any one of Embodiments 1-22, wherein the nucleic acid molecule is RNase-resistant.

Embodiment 24. A pharmaceutical composition comprising a therapeutically effective amount of the nucleic acid molecule of any one of Embodiments 1-22 and one or more additives or excipients.

Embodiment 25. The pharmaceutical composition of Embodiment 24, wherein the pharmaceutical composition is a composition for inhalation.

Embodiment 26. The pharmaceutical composition of Embodiment 25, wherein the pharmaceutical composition is in form of an aerosol, a nasal spray, an inhaler or a mouth spray.

Embodiment 27. The pharmaceutical composition of Embodiment 26, wherein the aerosol is a sea salt aerosol.

Embodiment 28. The pharmaceutical composition of Embodiment 24, wherein the pharmaceutical composition is a composition for intratumoral administration.

Embodiment 29. The pharmaceutical composition of Embodiment 24, wherein the pharmaceutical composition is a composition for intranodal administration.

Embodiment 30. The pharmaceutical composition of Embodiment 24, wherein the pharmaceutical composition is a composition for intravenous administration.

Embodiment 31. The pharmaceutical composition of Embodiment 24, wherein the pharmaceutical composition is a composition for administration through lymphatic transport.

Embodiment 32. The pharmaceutical composition of Embodiment 24, wherein the pharmaceutical composition further comprises a therapeutic agent.

Embodiment 33. The pharmaceutical composition of Embodiment 32, wherein the therapeutic agent is a vaccine, an anti-inflammatory agent, an antibody, a short-acting beta agonist, an antipyretic agent or an immune booster.

Embodiment 34. A method of treating or preventing a respiratory tract infection in a subject in need thereof wherein the method comprises administering to the subject a therapeutically effective amount of the nucleic acid molecule of any one of Embodiments 1-33.

Embodiment 35. The method of Embodiment 34, wherein the respiratory tract infection is in the upper respiratory tract of the subject.

Embodiment 36. The method of Embodiment 34, wherein the respiratory tract infection is in the lower respiratory tract of the subject.

Embodiment 37. The method of anyone of Embodiments 34-36, wherein the respiratory tract infection is a SARS infection.

Embodiment 38. The method of Embodiment 37, wherein the respiratory tract infection is a SARS-CoV-2 infection.

Embodiment 39. The method of Embodiment 38, wherein the subject suffers from acute respiratory distress symptoms.

Embodiment 40. The method of anyone of Embodiments 34-39, wherein the nucleic acid molecule is administered by inhalation.

Embodiment 41. The method of Embodiment 40, wherein the nucleic acid molecule is in form of an aerosol, a nasal spray, an inhaler or a mouth spray.

Embodiment 42. The method of Embodiment 41, wherein the nucleic acid molecule is administered once or twice a day.

Embodiment 43. The method of any one of Embodiments 34-39, wherein the nucleic acid molecule is administered intratumorally.

Embodiment 44. The method of any one of Embodiments 34-39, wherein the nucleic acid molecule is administered intranodally.

Embodiment 45. The method of any one of Embodiments 34-39, wherein the nucleic acid molecule is administered intravenously.

Embodiment 46. The method of any one of Embodiments 34-39, wherein the nucleic acid molecule is administered through lymphatic transport.

Embodiment 47. The method of anyone of Embodiments 34-46, wherein the method further comprises administering to the subject one or more therapeutic agents for treating or preventing one or more respiratory diseases.

Embodiment 48. The method of Embodiment 47, wherein the one or more respiratory diseases comprise asthma, COPD, pneumonia, sinusitis, laryngitis, bronchitis, a chest infection, or a cold.

Embodiment 49. The method of Embodiment 48, wherein the nucleic acid molecule and the one or more therapeutic agents are administered separately.

Embodiment 50. The method of Embodiment 48, wherein the nucleic acid molecule and the one or more therapeutic agents are administered together.

Embodiment 51. A method of treating or preventing a respiratory tract infection in a subject in need thereof wherein the method comprises administering to the subject the pharmaceutical composition of Embodiment 24.

Embodiment 52. The method of Embodiment 51, wherein the respiratory tract infection is in the upper respiratory tract of the subject.

Embodiment 53. The method of Embodiment 51, wherein the respiratory tract infection is in the lower respiratory tract of the subject.

Embodiment 54. The method of anyone of Embodiments 51-53, wherein the respiratory tract infection is a SARS infection.

Embodiment 55. The method of Embodiment 54, wherein the respiratory tract infection is a SARS-CoV-2 infection.

Embodiment 56. The method of Embodiment 55, wherein the subject suffers from acute respiratory distress symptoms.

Embodiment 57. The method of anyone of Embodiments 51-56, wherein the pharmaceutical composition is administered by inhalation.

Embodiment 58. The method of Embodiment 57, wherein the pharmaceutical composition is in form of an aerosol, a nasal spray, an inhaler or a mouth spray.

Embodiment 59. The method of Embodiment 58, wherein the pharmaceutical composition is administered once or twice a day.

Embodiment 60. The method of any one of Embodiments 51-56, wherein the pharmaceutical composition is administered intratumorally.

Embodiment 61. The method of any one of Embodiments 51-56, wherein the pharmaceutical composition is administered intranodally.

Embodiment 62. The method of any one of Embodiments 51-56, wherein the pharmaceutical composition is administered intravenously.

Embodiment 63. The method of any one of Embodiments 51-56, wherein the pharmaceutical composition is administered through lymphatic transport.

Embodiment 64. The method of anyone of Embodiments 51-63, wherein the method further comprises administering to the subject one or more therapeutic agents for treating or preventing one or more respiratory diseases.

Embodiment 65. The method of Embodiment 64, wherein the one or more respiratory diseases comprise asthma, COPD, pneumonia, sinusitis, laryngitis, bronchitis, a chest infection, or a cold.

Embodiment 66. The method of Embodiment 65, wherein the pharmaceutical composition and the one or more therapeutic agents are administered separately.

Embodiment 67. The method of Embodiment 65, wherein the pharmaceutical composition and the one or more therapeutic agents are administered together.

### EXAMPLES

### Example 1: Design of Multivalent Aptamers

### Background

More than hundreds of potential COVID-19 treatments have been tested in clinical trials, including drugs already used for other indications, such as Ebola virus disease, HIV/AIDS disease, malaria, inflammation and autoimmune disease, experimental antiviral compounds or vaccines previously validated in animal model, and convalescent plasma from recovered COVID-19 patients. However, no safe and effective drug treatments for the disease have been readily identified.

SARS-CoV-2 infection starts with its S protein binding to a target receptor ACE2. Similar to the SARS-CoV that caused the outbreak of SARS in 2003, SARS-CoV-2 binding to host cells is achieved through the interaction of its transmembrane spike glycoprotein with host cell surface receptor ACE2 on the host cell surface. Following receptor recognition and membrane fusion, the virus genome with its nucleocapsid is released into the cytoplasm of the host cells for subsequent viral replication and eventual new virion budding that is ready for infecting other host cells. The trimeric S protein mediates viral entry and elicits an immune response. Therefore, the S protein is the main target for diagnosis, treatment and vaccination. The S protein comprises two functional subunits responsible for binding to the host cell receptor (S1 subunit) and fusion of the viral and cellular membrane (S2 subunit). The key functional domain—receptor-binding domain (RBD)—is located in the distal S1 subunit and contributes to stabilization of pre-fusion state of the membrane-anchored S2 subunit that contains the fusion machinery. The RBD is recognized by the extracellular peptide domain of ACE2 receptor mainly through polar residue. Different coronaviruses use distinct RBDs within the S1 subunit to recognize a variety of attachment and entry receptors, depending on the viral species. It has been speculated that enhanced binding affinity between the RBD of SARS-CoV-2 and ACE2 is correlated with increased virus transmissibility and disease severity in humans.

Neutralizing agents can block viral entry by specifically targeting the RBD of the viral S protein. In the absence of a prophylactic vaccine, the first defense line against viruses would be to block the initial viral entry by employing neutralizing agents with broad breadth to avoid possible viral escape. Despite of the different binding affinity to human receptors, the sequence of SARS-CoV-2 RBD is relatively conserved in SARS-CoVs, such as 73.33% similarity with SARS-CoV RBD, 75.71% similarity with SARS-bat RBD, 74.29% similarity with SRAR-civet, suggesting the RBD is a critical target for the development of therapeutics and vaccines against SARS-CoVs. Additionally, although SARS-CoV-2 and SARS-CoV reside in separate branches of the corona virus family tree, 3D crystal structure analysis has revealed that the overall ACE2-binding mode of the SARS-CoV-2 RBD is nearly identical to that of the SARS-CoV RBD. Antigenic epitopes on SARS and SARS2 are only partially conserved. Consequently, and not surprisingly, most antibodies developed against SARS are not cross reactive to and do not neutralize SARS2. Limited exceptions have been described, most recently a human mAb 47D11 isolated from human IgG transgenic mice targeted the SARS-CoV RBD and neutralized SARS-CoV and SARS-CoV-2 with IC50 values of 0.19 and 0.57 μg/ml, respectively. In addition, another human monoclonal antibody S309 was isolated from memory B cells of an individual who was infected with SARS-CoV in 2003. The S309 neutralized authentic SARS-CoV-2 (2019n-CoV/USA_WA1-2020) with an IC50 of 79 ng/ml.

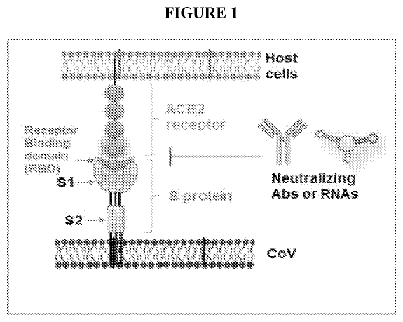

RNA-based neutralizing therapeutics provide an attractive technology approach due to their rational design and relative speed of development compared to conventional strategies. As a unique class of biomolecules, RNA aptamers or “chemical antibodies” not only possess the flexibility of small molecules, allowing them to access binding sites that may not be accessible to larger Abs, but also possess the high specificity of Abs, allowing targeted therapy that cannot be achieved with small drug molecules. They can be quickly evolved in the test tube and have exquisite binding affinity and selectivity to their target proteins. Rapid in vitro systematic enrichment of ligands by exponential enrichment (SELEX) technology underpins the speed of aptamer development and provides the potential for rapid ‘catch up’ with novel viral mutation, and could fill the absence of fast-track vaccines. Aptamers can serve as direct antagonists for blocking the interaction of disease-associated targets, and in addition are adaptable as target-specific carriers for guiding other therapeutic agents. Because of their fully synthetic nature, aptamers are readily amenable to molecular engineering approaches, including generation of oligomers, conjugates, and other multi-functional derivatives. Given that the SARS-CoV-2 S protein forms a homotrimer, presenting three closely spaced RBDs, aptamer multimerization is a particularly attractive opportunity to maximize binding avidity and half-life of the aptamer-RBD complex (FIG. 1). Furthermore, oligonucleotide aptamers are adaptable to inhaled delivery.

Inhaled RNAs offer a unique opportunity to address SARS-CoV-2 neutralization interventions. Similar to influenza and corona viruses that cause common colds or cold-like symptoms, both SARS-CoV and SARS-CoV-2 start their infection in the upper and lower respiratory tract which are widely recognized as the primary tissue site of viral attachment and entry. High expression levels of ACE2 receptors on luminal facing ciliated epithelial cells in the lower respiratory tract are considered the major binding interface and in addition recent studies have also identified goblet cells and ciliated epithelial cells in the upper respiratory tract as likely initial infection points in COVID-19 patients. SARS-CoV-2 lower respiratory infection can progress deep into the lungs and, once the infection goes beyond the protective lining of the airway, the risk of inflammatory cell accumulation and pulmonary fluid release significantly increases, resulting in acute respiratory distress symptom (ARDS). Viral spread beyond the lungs is well documented and can lead to multi-organ failure.

Given its physiological characteristics such as the massive surface area of the alveolar region, the abundant vasculature, the intensive capillary bed, and thin air-blood barriers, the lung is an attractive target for inhalation therapy, allowing faster drug uptake, less dosing and minimal systemic toxicity. The size and charge of nucleic acid therapeutics, which can be delivered in simple saline solution and independent of any special formulation, make them an ideal choice for inhaled drug delivery. Inhaled nucleic acids have proven to effectively target the pulmonary epithelium for the treatment of pulmonary diseases like SASR-CoV, asthma, cystic fibrosis (CF) and airway hyper-responsiveness (AHR). For example, in a rhesus macaque model, intratracheal administration of two anti-SARS-CoV siRNA cocktail treatments relieved SARS-CoV infection-induced fever, suppressed viral loads and protected lung from severe acute diffuse alveolar damage.¹⁷ Additionally, an aerosol delivered antisense oligonucleotides (ASO) targeting IL-4Rα reduced IL-4Rα surface protein expression on pulmonary epithelial cells as well as lung dendritic cells and alveolar macrophages and also decreased allergen-induced lung inflammation.¹⁸ ASOs delivered via aerosol was distributed into lung airway epithelial cells and systemic bio-availability was minimal. The inhaled ASOs were well-tolerated in mouse and non-human primate animal models at doses up to 15 and 50 mg/kg/exposure respectively.²² Collectively, these previous studies support the clinical feasibility of inhaled RNAs in the treatment of SARS-CoV-2-associated lung diseases. The inhaled RNA would also be well suited for combination therapy with other classes of drugs.

The goal of our study is to develop safe and effective, inhaled RNA aptamer therapeutics that provide immediate viral neutralizing effect, both for pre-exposure prophylaxis and post-exposure treatment, against SARS-CoV-2 and future SARS-CoV strains. To achieve our goal, we identify a panel of antagonistic RNA aptamers against the SARS-CoV-2 S RBD and explore their cross-reactive combinatorial use for maximal viral neutralization potency and breadth, including multivalent aptamers and cocktailed aptamers.

### Innovation

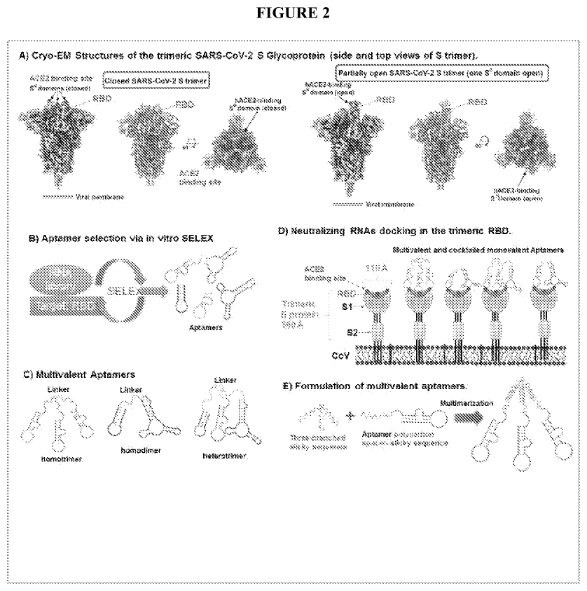

In addition to their utility as stand-alone antagonists, we explore multivalent aptamers (FIG. 2D) to block SARS-CoV-2 entry, seeking to provide maximal viral neutralization potency and breadth.

We use a non-covalent conjugation strategy allowing monovalent aptamers to serve as easily exchangeable building blocks for facile RNA multimerization (FIG. 2E).

Compared to systemic delivery, pulmonary delivery of RNA aptamers via inhalation offers several key advantages for SARS-CoVs associated respiratory diseases, including a noninvasive intervention, immediate drug availability to the tissue site of viral entry, high flexibility in administration schedule (either pre- or post-exposure treatment), and limited systemic side effect.

RNA aptamer-based technology can also offer the potential of greatly expedited future clinical development due to fast in vitro selection procedure, easy chemical manufacturing, excellent thermal stability and lack of immunogenicity.

### Multimerization for Improving Aptamer Avidity and Pharmacokinetic Properties

There are several major advantages for using a multivalent aptamer. First, a multivalent approach has been widely used to convert a low-affinity monovalent ligand into one with high avidity and increased cell binding properties. Second, multivalency can significantly reduce the ligand off-rate because only the simultaneous dissociation of all monomeric ligand from their receptors can result in the multivalent ligand completely dissociating from the target. Third, due to changes in size and lipophilicity, a multivalent approach may also improve pharmacokinetic performance, especially in cases where clearance properties and excretion rate are not optimal for the monovalent version. In our preliminary studies, a bivalent gp120 aptamer showed significantly improved binding affinity, cellular uptakes and serum stability, therefore encouraging us to prepare a multivalent aptamer containing an appropriate linker to achieve high avidity for SARS-CoV-2 S protein that naturally resembles a homotrimer protruding from the viral surface (FIG. 2). Such multivalent aptamer would improve RNA avidity and pharmacokinetic properties, consequently increasing the neutralization potency.

### Identification of High-Affinity RNA Aptamers Targeting the RBD of SARS-CoV-2 S Protein

RNase-resistant RNA aptamers are selected through protein-based SELEX. We use the SELEX procedure to identify RNase-resistant RNA aptamers against the RBD of SARS-CoV-2 S protein (Arg319-Phe541, functional binding to human ACE2, Cat #: 40592-V08B, Sino Biological). We perform seven selection rounds containing both positive and negative selection targets, and use barcode Illumina HST to identify the individual aptamer sequences for RNA pools from selection rounds 0-7. A panel of aptamers is obtained and representative RNAs are synthesized for further characterization.

### Characterization of Binding Affinity and Specificity of Selected Aptamers

A gel shift assay and surface plasmon resonance (openSPR®, Nicoya) are conducted to determine the binding affinity and target specificity of selected aptamers. Various recombinant full-length S proteins or the RBDs of SARS-CoV or SARS-CoV-2 (Sino Biological) will be incubated with P³² 5′ end-labeled, refolded RNA aptamers at various work concentrations (0-800 nM) and analyzed by electrophoresis. The dissociation constants of aptamers to its cognate target will be calculated using non-linear curve regression with a Graph Pad Prism 8.0. We will also conduct a competition assay to determine which aptamers compete with ACE2 to bind to the RBD of SARA-CoV-2 S protein. We will conduct flow cytometry and confocal microscopy to determine the extent to which the selected aptamers specifically bind to target-expressing cells. HeLa cells will be transfected with plasmids encoding SARS-CoV S or SARS-CoV-2 S C-terminally fused to GFP using lipofectamine 2000 (Invitrogen). We will label the aptamers with fluorescent dye (Life Technologies) and subsequently incubate the experimental RNAs at various work concentrations (0-800 nM) with target-expressing HeLa cells. After incubation, we will wash and resuspend cells in DPBS for flow cytometric analysis. To visualize specific cellular recognition and localization, we will perform live-cell Z-axis confocal microscopy. Unrelated aptamers or non-transfected HeLa cells will be used as negative controls.

### Determining the Antagonistic Function of the Selected Aptamers

We will assess which selected RBD aptamers functionally antagonize the interaction of S protein and ACE2. We will use a SARS-CoV-2 Spike-ACE2 inhibitor screening assay kit based on the immobilized SARS-CoV-2 S protein using chemiluminescent detection (BPS Bioscience) per the manufacturer's protocol. Additionally, we will conduct flow cytometry to measure the ability of the selected aptamers to block the interaction of S protein and ACE2. HeLa cells will be transfected with plasmids encoding human ACE2-C-terminally fused to GFP. The RBD of SARS-CoV and SARS-CoV-2 will be pre-incubated with fluorescently labeled, refolded experimental RNAs at a constant concentration (100 nM) for 1 hour. Subsequently, ACE2-expressing HeLa cells will be incubated with the mixture of RNA aptamer and RBD protein, and subjected to flow cytometry.

### Example 2: Development of Combinatorial RBD Aptamers to Neutralize Viral Infection In Vitro

To rationally design multivalent aptamers, given that the SARS-CoV-2 S protein naturally resembles a homotrimer protruding from the viral surface, we engineer multivalent aptamers to increase avidity and achieve synergetic effect (FIG. 2C-E). The linker length and orientation of the aptamers must be taken into account when designing trivalent aptamers, as the spatial distance is important for enabling maximal blockade and effective neutralization. The SARS-CoV-2 S ectodomain is a 160-A-long-trimers with a triangular cross-section (˜110-A in width). We design a three-branched GC rich “sticky sequence” to connect to three aptamers (FIG. 2E). Each aptamer contains a complementary sticky sequence, thereby allowing this three-branched linker to be annealed through Watson-Crick based pairing by simple mixing. By adjusting the length of the sticky sequence in each branch (˜34 A/10 bp, 10 bp per helical turn), we position the three aptamer components in the correct orientation to interact with the ACE2 binding site in the trimeric RBD. To further increase the likelihood that the multivalent aptamers simultaneously bind three binding sites on the RBD at the appropriate distance (in a range of 110 A), we insert a flexible polycarbon spacer between the aptamer and the sticky sequence (3 A per C3), which provides molecular flexibility without interfering with correct folding or target-driven spatial orientation of the aptamers. Thus, rationally combining double-stranded sticky sequences and flexible polycarbon spacers offers a framework for presenting aptamers in various configuration, increasing the likelihood that a give aptamer against a target can be presented in the necessary manner to achieve its function. We design 10 trivalent aptamers, including 6 homotrimers and 4 heterotrimers for three selected top aptamers, with two putative linker lengths (87.8 and 117.8 A) that are calculated from the total distance of one sticky sequence plus one polycarbon spacer, and two unrelated trivalent controls. We confirm the formulation of these multivalent RNA using gel electrophoresis.

### Determination of the Binding Affinity, Target Specificity, RNA Stability and Antagonistic Function of Multivalent Aptamers

We determine the ability of the designed multivalent aptamers to bind the RBD and full-length S protein of SARS-CoV or SARS-CoV-2 by gel shift assay and flow cytometry. We use a SARS-CoV-2 Spike-ACE2 inhibitor screening assay kit and flow cytometry-based receptor-binding inhibition assay to determine the antagonistic ability of multivalent aptamers. The dissociation constants, the target specificity, RNA stability in mouse serum and antagonistic function of multivalent aptamers are calculated and compared with monovalent aptamers.

### Determination of Viral Neutralization Activity of the RNAs In Vitro

To assess the effect of the designed multivalent, cocktailed or monovalent aptamers on the viral suppression and specificity, we conduct two assays. In the first assay (as a post-exposure intervention), we firstly infect ACE2-expressing cell line (Vero E6) with SARS-CoV or SARS-CoV-2 respectively for 24 hours. Subsequently, we incubate the experimental RNAs (0-800 nM) with the infected Vero E6 cells. Two unrelated monovalent and trivalent aptamers are included as negative control. One published mAb S309 (0-200 ng/mL) is included as positive control. We collect total RNA and aliquots of the cell-free media at different time points post treatment (24-72 hr). We quantify the viral RNA by probe specific Taqman PCR and viral protein by Spike ELISA (Sino Biological). In the second assay (as a pre-exposure prophylactic intervention), we firstly pre-incubate viruses with these experimental RNAs (0-800 nM) or S309 (0-200 ng/ml) for 1 hr. Subsequently the treated viruses are used for infecting Vero E6. The infectivity of the RNA-treated viruses is measured by Taqman PCR and ELISA as described. The viral neutralization activity of multivalent aptamers is compared with monovalent or cocktailed format. The half maximal inhibitory concentrations (IC50 value) is determined using related inhibitory ability (GraphPad Prism version 8). The top combinatorial modality is further tested in vivo.

### Example 3: Pre-Exposure Prophylaxis and Post-Exposure Treatment In Vivo

### Establishment of SARS-CoV-2 Infection in a Human ACE2-Transgenic Mouse Model

Transgenic mice that express human ACE2 in airway and other epithelia have been developed for studying pathogenesis of SARS-CoVs. K18-hACE2 transgenic mice can develop a rapid lethal infection after intranasal inoculation with a human SARS-CoV.36 Pre-treatment with a human anti-SARS-CoV mAb prevents clinical disease, death and weight loss. Infection begins in airway epithelia, with subsequently alveolar involvement and extra-pulmonary virus spread. Infection also results in macrophage and lymphocyte infiltration in the lungs and up-regulation of pro-inflammatory cytokines in both the lung and the brain. Therefore, K18-hACE2 model may represent an effective surrogate for COVID-19 patients. We establish SARS-CoV-2 infection in K18-hACE2 transgenic mice (JAX® stock #034860). The hACE2 mice is inoculated intranasally with SARS-CoV-2 at a dosage of 2×103 PFUs in 20 μL inoculum volume per mouse after mice will be lightly anesthetized with halothane.

### Evaluating the Ability of the Top Neutralizing RNAs to Inhibit SARS-CoV-2 Replication In Vivo

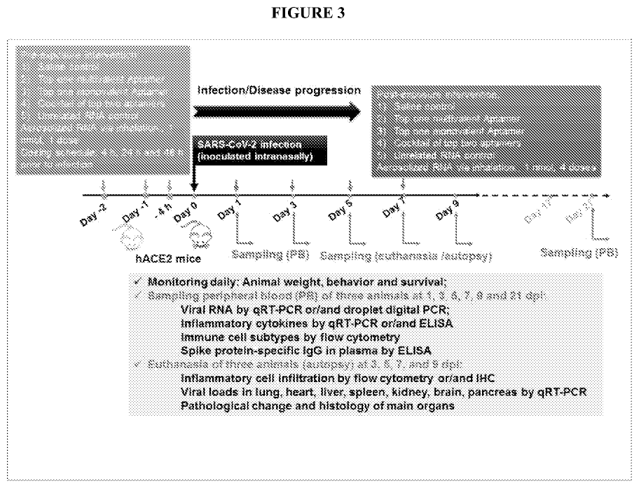

As shown in FIG. 3, we design two experiments to determine the neutralizing activity of aptamers as a pre- or a post-exposure intervention approach, respectively. To calculate the number of the experimental mice needed for statistical power, we perform Bonferroni correction analysis to adjust for multiple comparisons under the following conditions. When choosing nominal type I error at 0.05, with Bonferroni correction, we set a, at 0.05/5=0.01. With 16 mice per experimental condition, we have 85% power to detect changes at an effect size of 3 with a 1-sided test, using a two-sample t-test. We consider sex as a biological variable; because response to SARS-CoV-2 infection and RNA treatment may vary based on sex. We use equal number of female and male mice to evaluate treatment. In addition to mock-infection mouse control (n=8), we need total 128 infected-hACE2 mice (64 males and 64 females, n=4 females and 4 males per group) for all 16 groups (FIG. 2).

In the pre-exposure intervention assay (11 groups): 1) Saline control (inhalation); 2) top one multivalent aptamer (inhalation, 1 nmol, 1 dose); 3) top one monovalent aptamer (inhalation, 1 nmol, 1 dose); 4) cocktail of top two monovalent aptamers (inhalation, 1 nmol, 1 dose); and 5) Unrelated RNA (inhalation, 1 nmol, 1 dose). Condition 2), 3), and 4) contain 24 animals for three different dosing schedules: 4 h, 24 h and 48 h prior to SARS-CoV-2 infection, respectively.

In the post-exposure intervention assay (5 groups): 1) Saline control; 2) top one multivalent aptamer (inhalation, 1 nmol, 4 doses); 3) top one monovalent aptamer (inhalation, 1 nmol, 4 doses); 4) cocktail of top two monovalent aptamers (inhalation, 1 nmol, 4 doses); and 5) Unrelated RNA (inhalation, 1 nmol, 4 doses).

ACE2-transgenic mice receive aerosolized doses of the experimental RNA's and controls as scheduled in FIG. 2. For aerosol dosing, RNA suspended in DPBS with Ca²⁺ and Mg²⁺ (Sigma) is delivered via inhalation using a nose-only delivery system. A Lovelace nebulizer system is used to generate RNA aerosol at a flow rate of 1 liter/min. The estimated particle size of aerosolized RNA in the nose-only system is approximately in 1-2 μm range, which may provide uniform and efficient delivery to the deep lung airspace. The exposure chamber is equilibrated with the RNA aerosol suspension for 5 min before mice is placed in restrain tubes and attached to the chamber. The restrained mice is treated with aerosolized RNA, control RNA or saline (vehicle control) for 30 min per exposure. Based on our previous HIV-1 study 25, we propose to use 1 nmol of RNA (estimated inhalable dose of 0.36 and 1.1 mg/kg when 30 nt monovalent aptamer and 100 nt multivalent RNA are applied, respectively) per individual animal in the present study.

We measure daily the animal weight, monitor the behaviour and survival rate. We collect peripheral blood (PB) samples at 1, 3, 5, 7, 9 and 21 dpi (day post infection) (3 animals from each group). We isolate cells and total RNA for various purposes. We monitor infection progression by detecting PB viral loads via Taqman qPCR and droplet digital (dd)PCR, CD4 and CD8 counts by flow cytometry, desired gene expression in PB by qRT-PCR, including: inflammatory cytokines (e.g., IL-1β, IL-6, IL-8, TNF-α, INF-γ) and key immune mediator genes (e.g., PD-1, PD-Li, CXCR4, NF-kB, Arginase 1). We isolate mouse serum for quantification of inflammatory cytokines by Luminex® assay (e.g., IL-1β, IL-6, IL-8, IL-10, TNF-α, INF-γ, MMPs) and anti-Spike IgG antibody by ELISA. At 3, 5, 7 and 9 dpi, we sacrifice animals (3 mice from each group) to collect various tissues (e.g., PB, lung, heart, liver, spleen, kidney, brain, pancreas) and bronchoalveolar lavage (BAL). In particular, mouse lung is lavaged to collect BAL fluid samples. The recovered airway cells in BAL are assayed for viral RNA expression by qRT-PCR or ddPCR; while cell-free lavage fluid is assayed for inflammatory cytokine level by ELISA.

To investigate inflammatory cell infiltration, immunohistochemistry is carried out to identify CD14+ monocytes/macrophages, CD16+ granulocytes/neutrophil, CD3+ T-lymphocytes, and CD19+ B-lymphocytes. We stain cells from PB and lung with corresponding fluorochrome-conjugated antibodies for immune cell subtype analyses by flow cytometry (e.g., CD3, CD4, CD8, CD10, CD11b, CD14, CD16b, CD25, CD68, CD69, CD80, and CD163). We perform histopathological analysis to evaluate infection-induced tissue injury under microscopy (lung, heart, liver, spleen, kidney, brain, and pancreas). Specifically, mouse lung is fixed, parafinized, sectioned and stained with hematoxylin and eosin for examining cell infiltration and pathological change. Viral RNA in total RNA from different organs is measured by Taqman qPCR and ddPCR. We compare viral loads in PB and lung among experimental groups; their suppression is indicative of efficacy. We compare the antiviral efficacy of multivalent RNA versus monovalent RNA.

### Example 4: Effect of Delivery of Neutralizing Aptamers by Inhalation as Therapeutics Against SARS-CoV2

### SELEX Protocol

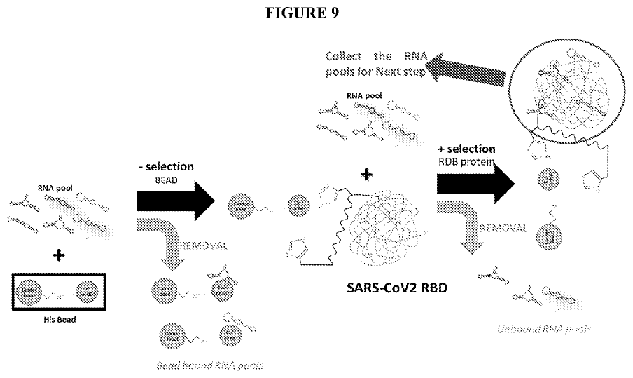

2′-Fluoropyrimidine-modified RNA aptamers that selectively bind to recombinant Spike protein were selected using an in vitro SELEX procedure. As a target for SELEX, the RBD domain of the spike protein was modified with a six-histidine (His6) tag to immobilize to beads, and expressed in HEK293 cells. An RNA aptamer library pool was first incubated with agarose beads to remove non-specific binders, and the resulting supernatant was incubated with the His6-spike target protein for positive selection. The aptamers bound to the spike protein were amplified by PCR and in vitro transcription, as depicted in FIG. 9. Clustering analysis of RNA libraries was used to identify related sequence groups. After alignment of the top 100 unique sequences, several sequence groups were identified. The representative sequences identified for each group, and their reads and frequencies are shown in Table 1 below, are listed. Only the 40-nt random sequences of the RNA core regions (5′-3′) are indicated.

TABLE 1
Selection and Identification of Aptamers by SELEX

| | | Reads | | Enrichment |
|---|---|---|---|---|
| SEQ ID | | in Top | % | ratio |
| NO: | seq (5′ to 3′) | 50 | Frequency | 4th to 5th |
| 1 | CCTTTGACCATAGGAGGCAAGTCTTATGTTCAAACTGGTC | 6604 | 23 | 10.65 |
| 2 | TCTTGAWCGCCCTGAAGCGGCCTTTTCACGAGGGTACC | 3069 | 11 | 6.49 |
| 3 | GTAGAACTTGGAGAGCCACCTTCTAAGGCGCAGTCACACG | 1295 | 4 | 8.19 |
| 4 | TTGAACAGAACCATTGTCCTTCAATGCCTAGCAACCTGTC | 839 | 3 | 7.44 |
| 5 | GTATAAACTTAGCAGACTTTACACGGCACACAAAGCTTTC | 657 | 2 | 12.08 |
| 6 | TGGTGAGTAMCATAGGTGCATACTCTACCTTTGCTCATC | 3000 | 12 | 2.23 |
| 7 | TT GAGAGCGGATGCTAGTTTAGTTATAGCCTCCGACTCTC | 1759 | 7 | 3.33 |
| 8 | ATCATAAATCATATAACATGCWATCTTTATGGTTTATC | 1181 | 5 | 2.64 |
| 9 | TTACCTTAACATACTGCAGTACTTAAGAGGAGGATGGCAG | 714 | 3 | 3.18 |
| 10 | TAAACGATCGAGACCCTTTAGGTGCAGCATTATCTGCGTA | 460 | 2 | 3.08 |
| 11 | GAACGGGTAAACAGCTAATGTCGATTTCTCTTTGCCCGCTC | 3696 | 15 | 3.37 |

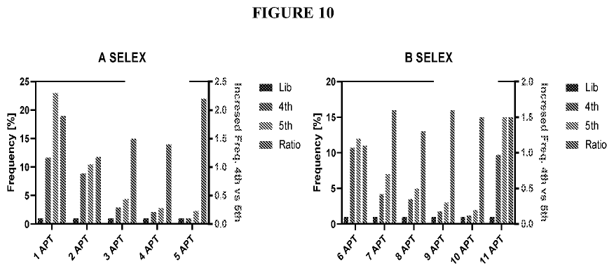

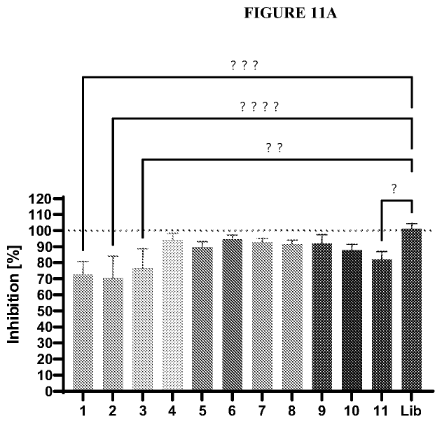

FIG. 10 shows the increase in the frequency of specific nucleic acid compound groups with each round of selection via SELEX. The molecular enrichment of each group of nucleic acid compounds is shown in the initial library (Lib), and after the 4th round and the 5th round of selection. The initial library contained 40 random nucleotide sequences that were carefully designed and selected to maximize the diversity and coverage of the aptamer library, while ensuring aptamer stability, functionality, and high affinity and specificity for a wide range of targets.

The percent frequency of each group of nucleic acid compounds at each selection round was calculated by dividing the reads of each group by the total reads of the top 1000 unique sequences.

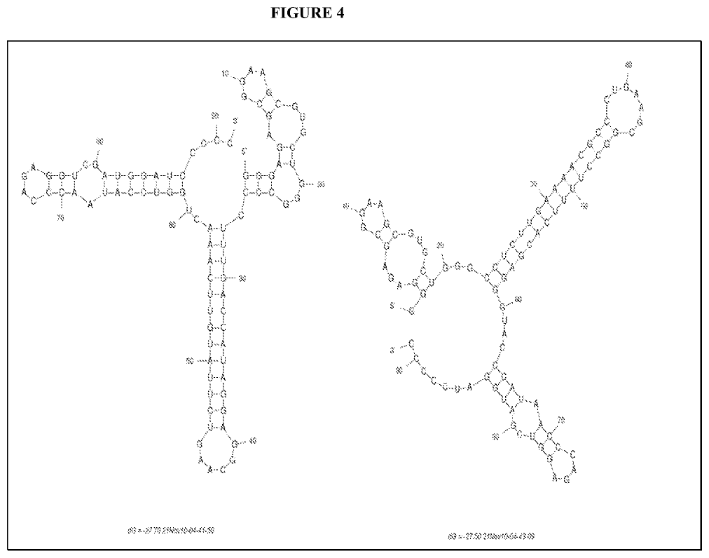

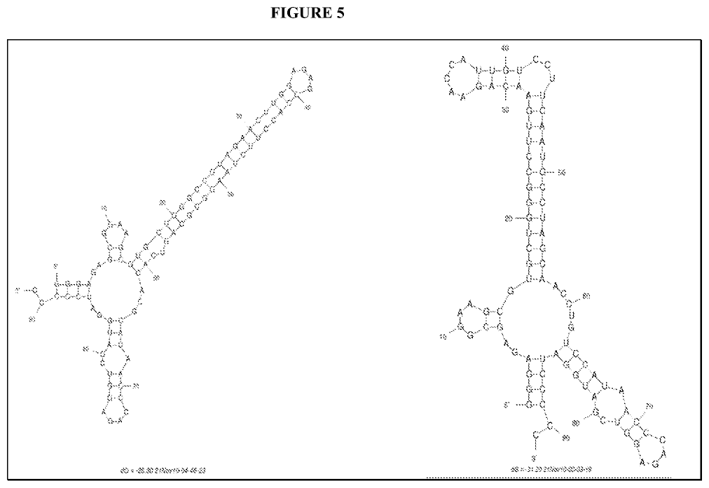

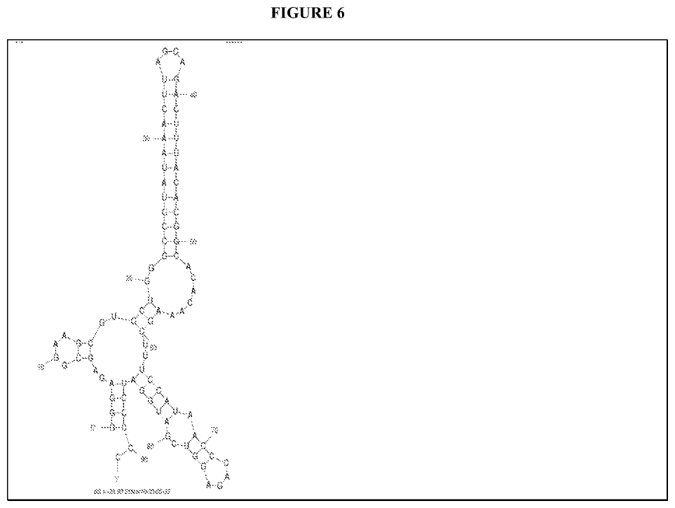

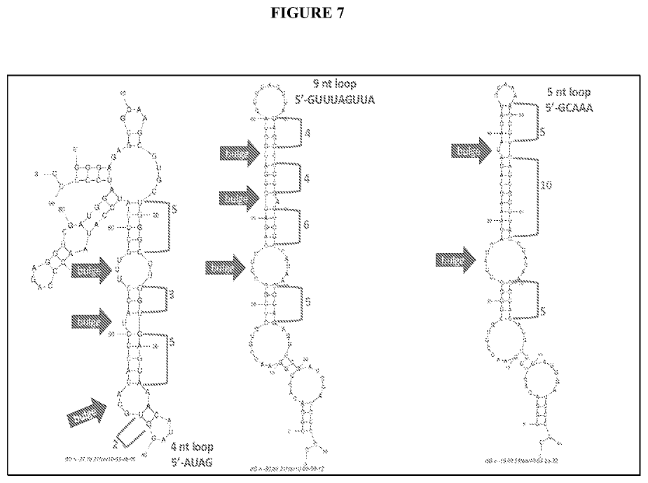

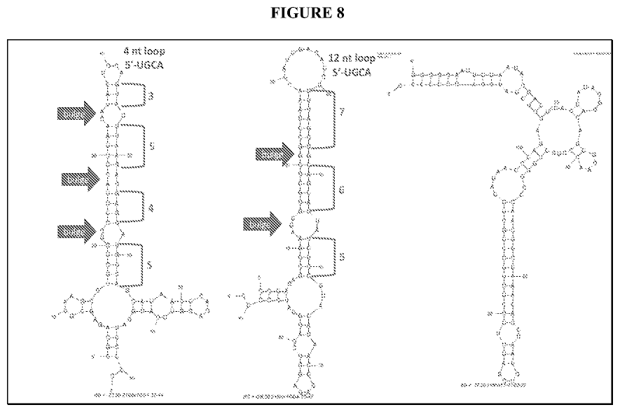

The structure of each identified aptamer is shown in FIGS. 4-8.

### Effect of Candidate Aptamers on Neutralization of Spike Protein In Vitro

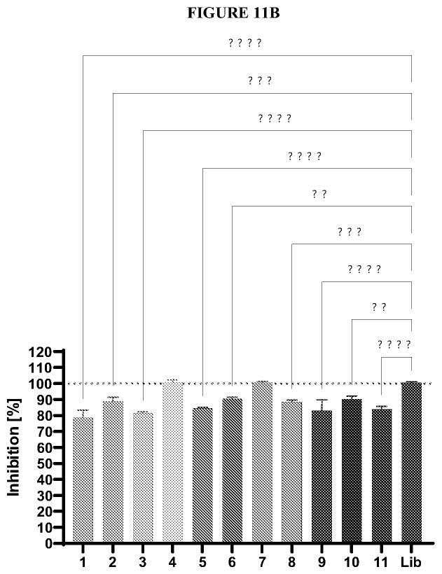

Candidate aptamers were tested for their ability to neutralize the S protein in vitro using the InvivoGen™ cell fusion assay between the Spike protein and the human ACE2 receptor, and detection by the SEAP detection reagent QUANTI-Blue™ Solution. The assay relies on the transfer of the adaptor molecule, MyD88, from a “donor cell line” to an “acceptor cell line” expressing an NF-kB-SEAP inducible reporter gene. Cell fusion is then readily assessable in the co-culture supernatant using the SEAP detection reagent, QUANTI-Blue Solution™. FIG. 11 shows the results of the neutralization assay with the candidate nucleic acid compounds of SEQ ID NOS: 1-11. Each bar corresponds to one of the candidate nucleic acid compounds of SEQ ID NOS: 1-11. The percentage of neutralization for each tested sample in cells expressing the original spike protein (A) or the omicron spike variant (B) was calculated based on the neutralization value for the original Library sample. The Data show that the aptamer of SEQ ID NO: 1 effectively neutralized the spike protein of the original SARS-CoV2 as well as the spike protein of the Omicron variant expressed in HEK293 cells.

### Development of Truncated Aptamers

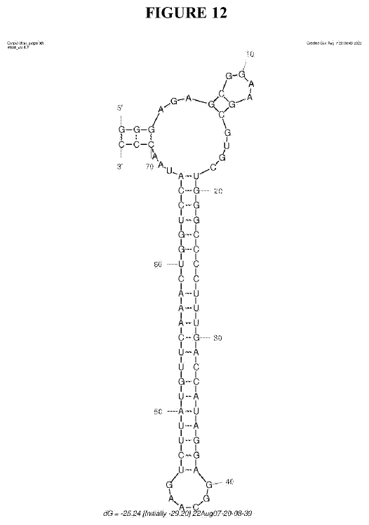

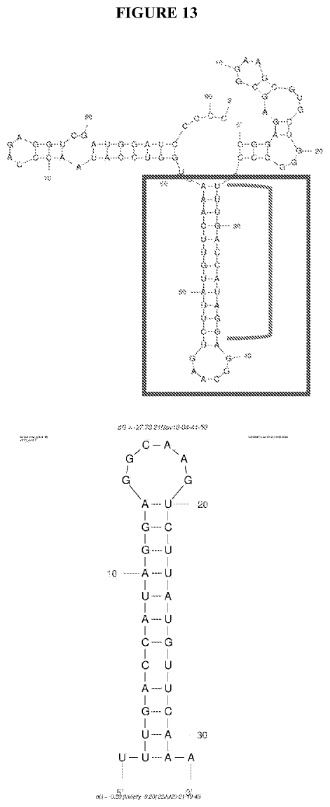

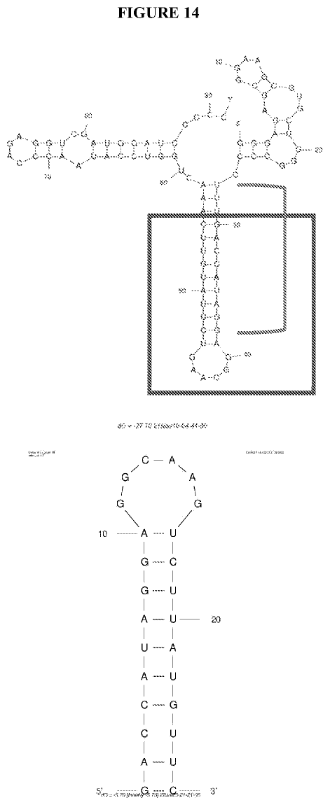

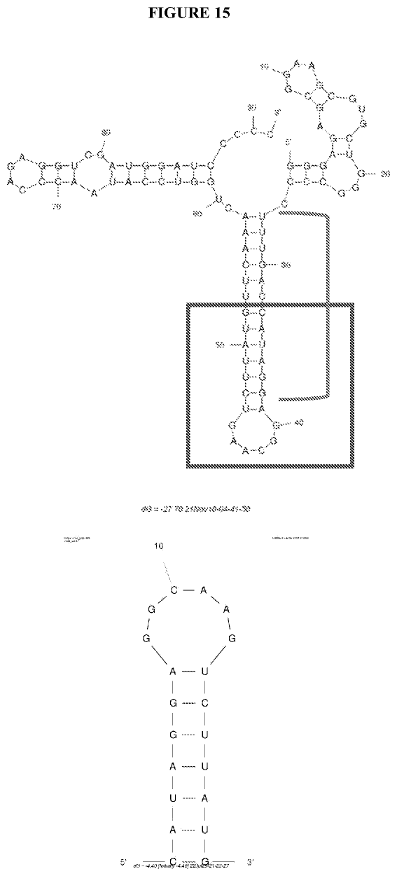

Using computational prediction, we truncated the aptamer of SEQ ID NO: 1 into the smallest functional units that were expected to maintain binding to the spike protein. We generated four truncated aptamers from the aptamer of SEQ ID NO: 1: a 72 nucleotide long aptamer of SEQ ID NO: 14; a 32 nucleotide long aptamer of SEQ ID NO: 15; a 26 nucleotide long aptamer of SEQ ID NO: 16; and a 20 nucleotide long aptamer of SEQ ID NO: 17. These truncated aptamers are shown in FIGS. 12-15.

### Evaluation of Binding Affinity

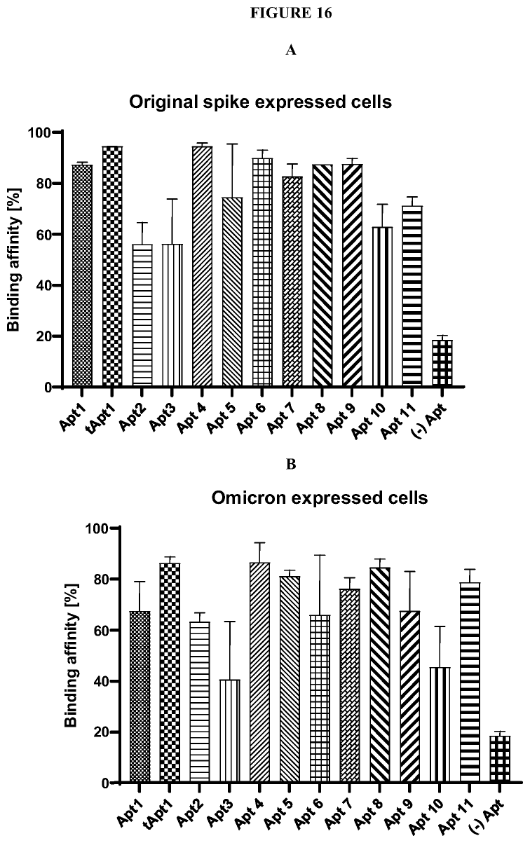

The candidate nucleic acid compounds of SEQ ID NOS: 1-11 were labeled with a fluorescent marker and their binding affinity toward the spike protein of the original SARS-CoV2 and the spike protein of the Omicron variant expressed in HEK 293 cells was measured by flow cytometry. FIG. 16 shows the ability of the selected nucleic acid compounds to bind to the SARS-CoV2 spike protein (A) or to the spike protein of the Omicron variant (B) expressed in HEK293 cells, as compared to a nucleic acid compound (−) that targets CD8. The amount of nucleic acid compound bound to the cells is represented as percentage of input RNA.

Flow cytometry analysis shows that the selected RNA aptamers are capable of selectively binding to the target SARS-CoV2 spike protein and to the spike protein of the Omicron variant expressed in HEK293 cells. Table 2 below shows the dissociation constant (KD) of the candidate nucleic acid compounds of SEQ ID NOS: 1-11 and the 72 nucleotide long aptamer of SEQ ID NO: 14 for the SARS-CoV2 spike protein and the spike protein of the Omicron variant.

TABLE 2
Binding Affinity of Candidate Aptamers

| | | | | | | | | | | | | | |
|---|---|---|---|---|---|---|---|---|---|---|---|---|---|
| | SEQ ID | SEQ ID | SEQ ID | SEQ ID | SEQ ID | SEQ ID | SEQ ID | SEQ ID | SEQ ID | SEQ ID | SEQ ID | SEQ ID | Control |
| [nM] | NO: 1 | NO: 14 | NO: 2 | NO: 3 | NO: 4 | NO: 5 | NO: 6 | NO: 7 | NO: 8 | NO: 9 | NO: 10 | NO: 11 | (−) Apt |

Spike

| | | | | | | | | | | | | | |
|---|---|---|---|---|---|---|---|---|---|---|---|---|---|
| Kd | 37.71 ± | 20.73 ± | 105.68 ± | 106.03 ± | 26.37 ± | 62.09 ± | 33.29 ± | 43.71 ± | 41.12 ± | 22.38 ± | 64.89 ± | 31.44 ± | 311.97 ± |
| | 0.12 | 1.46 | 18.35 | 48.74 | 2.44 | 33.17 | 1.40 | 5.09 | 2.66 | 5.01 | 1.25 | 0.04 | 33.63 |

Omicron

| | | | | | | | | | | | | | |
|---|---|---|---|---|---|---|---|---|---|---|---|---|---|
| Kd | 31.92 ± | 20.80 ± | 83.35 ± | 167.93 ± | 36.77 ± | 49.16 ± | 54.31 ± | 51.21 ± | 41.18 ± | 38.56 ± | 105.87 ± | 32.94 ± | 537.54 ± |
| | 2.53 | 3.18 | 12.19 | 99.59 | 2.47 | 0.15 | 14.87 | 6.06 | 7.40 | 6.01 | 29.80 | 3.64 | 72.26 |

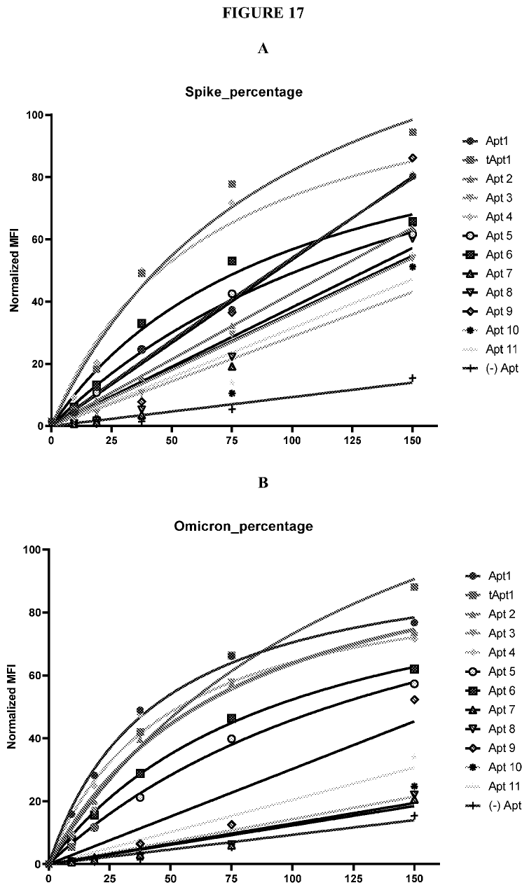

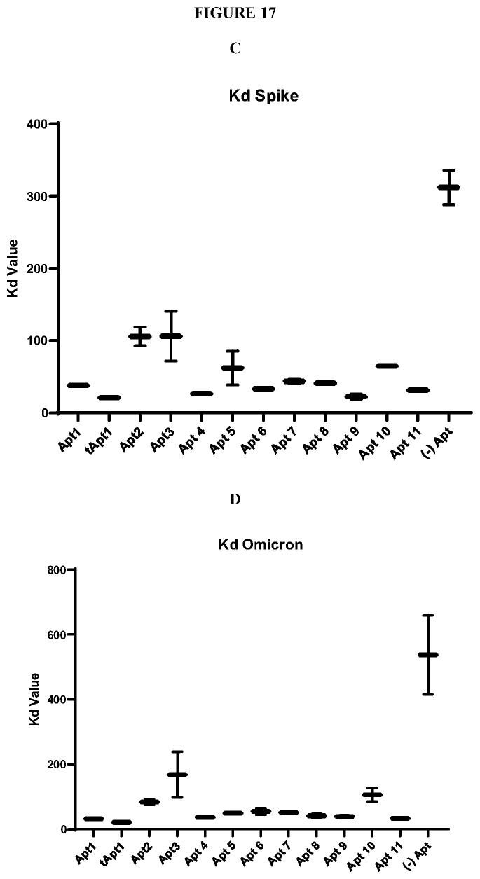

The dissociation constant (KD) of the 72 nucleotide long aptamer of SEQ ID NO: 14 for the SARS-CoV2 spike protein and the spike protein of the Omicron variant was 20.73 nmol/L and 20.80 nmol/L, respectively. See also FIG. 17. These data clearly demonstrate that the selected aptamers specifically bind to the spike target protein of both the SARS-Co-V2 and the Omicron variant. It is predicted that aerosol administration of the nucleic acid molecules provided herein results in robust and durable efficacy against SARS-CoV-2 both as pre- and post-exposure interventions, with significant antiviral activity, reduced viral resistance and a prolonged circulating time in vivo, thereby effectively neutralizing SARS-CoV-2 entry and infection.

### Expected Results and Alternative Approaches

Viruses are the products of natural evolution and dynamically mutate to adapt environment pressure. More contagious mutant strains of SARS-CoV-2 could become dominant across the world and cause re-infection among those who already have acquired antibodies in a previous infection. Fourteen mutations in the viral S protein (residues 1-681) have been found, including one high-frequency D614G (3577 in total 3908 count, −91.5%) at the far distal of S1 subunit, and one rare G476S (8 in total 3908 count, −0.2%) at the RBD (residues 329-521). Considering that the SARS-CoV-2 RBD is relatively conserved and shows a low mutation frequency, it is expected that the nucleic acid compounds will retain their binding affinity and antagonistic function to various strains of SARS-CoV-2.

### Example 5: Animal Studies

### Description of Procedures

Animal maintenance and care: Human ACE2 transgenic mice are purchased from the Jackson Laboratory (B6.Cg-Tg (K18-ACE2) 2Prlmn/J strain, #034860). Animal breeding, maintenance and experiment are performed in the Animal Care Facility at the City of Hope under the approved IACUC protocol. Animals are cared in the barrier facility of the City of Hope Animal Resources Center (ARC) institutional vivarium within a 14:10 light: dark cycle. Animals are maintained on sterile food and receive acidified water (pH 2.8-3.2). Prior to viral inoculation, animals are maintained under specific-pathogen-free husbandry conditions and acclimated to the Biosafety Level 3 (BSL3) laboratory prior to inoculation.

Establishment and Characterization of SARS-CoV-2 Infection in hACE2 Transgenic Mouse Model

Animal studies are performed in the BSL3 facility using HEPA-filtered isolators. Male and female K18-hACE2 mice are inoculated intranasally with the SARS-CoV-2 stock virus at a dosage of 2×10², 2×10³ or 1×104 plaque forming units, PFUs in 20 μL inoculum volume per mouse. The mice is lightly anesthetized with halothane (3 dosages, 6 females and 6 males per condition), and intranasally inoculated. Equal volume of PBS is used as mock-infection control. General anesthesia (Isofluorane) ensures safe stable introduction of the virus.

The infected mice are observed daily to record body weight, behavior, responsiveness to external stimuli and death, and clinical symptoms. At 1, 3, 5 and 7 day post infection (dpi), 3 animals per condition are euthanized by CO₂ inhalation and lymphoid organs are evaluated for the presence and relative levels of human immune cells by flow cytometry (CD45, CD3, CD4, and CD8, CD10, CD11b, CD14, CD16b, CD19, CD25, CD66b, CD68, CD69, CD80, CD163). Pathological features are characterized by pathological examination of autopsies, including virus replication in lung, pulmonary lesions, interstitial pneumonia, inflammatory cells infiltration, upregulation of pro-inflammatory cytokines, and/or production of anti-spike IgG antibody. Histopathological analysis is performed to evaluate infection-induced tissue injury under microscopy (e.g., lung, heart, liver, spleen, kidney, brain, and pancreas). Viral RNA in total RNA from different organs is measured by Taqman qPCR and ddPCR.

### In Vivo Effect of Inhaled Nucleic Acid Molecule Candidates on Viral Loads

Approximately 200 male/female K18-hACE2 mice are used for establishment of SARS-CoV-1 infection and functional evaluation of the therapeutic nucleic acid molecule compounds.

To compare the sensitivity of the developed RNA therapeutics between male and female mice, equal numbers of male mice and female mice are used to evaluate the nucleic acid molecule compounds. To examine whether the response to the various treatments vary by sex, we compare outcomes in males with females (e.g., plasma viral RNA levels, viral protein expression, bio-distribution of RNAs) within the various experimental groups as described above.

In this regard, transgenic mice that express the hACE2 receptor in airway and other epithelia have been developed for studying infection and pathogenesis of SARS-CoV or SARS-CoV-2.(1, 2) For example, K18-hACE2 transgenic mice can develop a rapid lethal infection after intranasal inoculation with a human strain of SARS-CoV.(1) hACE2 gene was driven by human cytokeratin 18 (K18) promoter in epithelial cells. Infection begins in airway epithelia, with subsequently alveolar involvement and extra-pulmonary virus spread to the brain. Infection also results in macrophage and lymphocyte infiltration in the lungs and up-regulation of pro-inflammatory cytokines in both the lung and the brain. Pre-treatment of K18-hACE2 mice with a human anti-SARS-CoV monoclonal antibody prevents clinical disease, death and weight loss. It is currently available in JAX®. Most recently, another hACE2 transgenic mouse model was applied for SARS-CoV-2 infection. hACE2 gene was driven by the mouse ACE2 promoter into the mouse genome and hACE2 gene was expressed in lung, heart, kidney and interstine. It recapitulates the major features and pathological consequence of SARS-CoV-2 infection, including weight loss, virus replication in lung, interstitial pneumonia, significant infiltration of lymphocytes and monocytes in alveolar interstitium, accumulation of macrophages in alveolar cavities, and production of SARS-CoV-2 Spike protein specific IgG antibody. Although it showed severe lung damage and systemic inflammatory reaction, degeneration and necrosis in many extrapulmonary organs, pro-inflammatory cytokine related study was not reported in this model. Additionally, compared to K18-hACE2 model, this model shows that hACE2 expression has a more limited tissue distribution and the permissiveness to SARS-CoV infection, and it does not develop lethal disease. Collectively, we will choose the well-established K18-hACE2 transgenic mouse model for our study.

### Minimization of Pain and Distress

Animals are anesthetized with halothane or isoflurane. Once the animal is initially anesthetized in the iso chamber, one drop of proparacaine is applied to the animal, which is then returned to the chamber to achieve the level of anesthesia necessary to perform the bleed. Animals are monitored closely for behavior indicating post-procedure complications from irradiation or IV injection of human hematopoietic cells at least two times in the 24 h post-injection and on a weekly basis during experimentation. Signs that the animals are dehydrated, hunched over, have ruffled fur, or reduced appetite are monitored. Veterinary care is administered in a timely manner.

### Euthanasia

Animals will be euthanized by C02 inhalation. Physiological parameters such as cessation of the heartbeat and respiration are used to confirm death. Animals are euthanized if they exhibit a loss of 20% body weight, fail to eat food or drink water, fail to make normal postural adjustment or display normal behavior, or if there are any signs of obvious stress. Animals that do not show signs of engraftment at 12 weeks or that lose engraftment any time after 12 weeks are euthanized by CO₂ inhalation. 2/10 animals in each cohort may fail to engraft. These methods are in accordance with the American Veterinary Medical Association (AVMA) Guidelines for the Euthanasia of Animals.

### Informal Sequence Listing

(5′ to 3′):
SEQ ID NO: 1
CCTTTGACCATAGGAGGCAAGTCTTATGTTCAAACTGGTC
(5′ to 3′):
SEQ ID NO: 2
TCTTGAAAACGCCCTGAAGCGGCCTTTTCACGAGGGTACC
(5′ to 3′):
SEQ ID NO: 3
GTAGAACTTGGAGAGCCACCTTCTAAGGCGCAGTCACACG
(5′ to 3′):
SEQ ID NO: 4
TTGAACAGAACCATTGTCCTTCAATGCCTAGCAACCTGTC
(5′ to 3′):
SEQ ID NO: 5
GTATAAACTTAGCAGACTTTACACGGCACACAAAGCTTTC
(5′ to 3′):
SEQ ID NO: 6
TGGTGAGTAAACATAGGTGCATACTCTACCTTTGCTCATC
(5′ to 3′):
SEQ ID NO: 7
TTGAGAGCGGATGCTAGTTTAGTTATAGCCTCCGACTCTC
(5′ to 3′):
SEQ ID NO: 8
ATCATAAATCATATAACATGCAAAATCTTTATGGTTTATC
(5′ to 3′):
SEQ ID NO: 9
TTACCTTAACATACTGCAGTACTTAAGAGGAGGATGGCAG
(5′ to 3′):
SEQ ID NO: 10
TAAACGATCGACCCTTTAGGTGCAGCAGCATTATCTGCGTA
(5′ to 3′):
SEQ ID NO: 11
GAACGGGTAAACAGCTAATGTCGATTTCTCTTTGCCCGCTC
SEQ ID NO: 12:
GGGAGAGCGGAAGCGUGCUGGGCCC
SEQ ID NO: 13:
CCCCCUAGGUAGCUGGAGACCCAAUAC
SEQ ID NO: 14:
GGG AGA GCG GAA GCG TGC TGG GCC CCT TTG ACC
ATA GGA GGC AAG TCT TAT GTT CAA ACT GGT CCA
TAA CCC
SEQ ID NO: 15:
UUU GAC CAU AGG AGG CAA GUC UUA UGU UCA AA
SEQ ID NO: 16:
GAC CAU AGG AGG CAA GUC UUA UGU UC
SEQ ID NO: 17:
CAU AGG AGG CAA GUC UUA UG.

### REFERENCES

1. BioCentury. COVID-19 clinical vaccines and therapies 2020. doi: https://www.biocentury.com/clinical-vaccines-and-therapies.
2. Kupferschmidt K, Cohen J. Race to find COVID-19 treatments accelerates. Science. 2020; 367(6485):1412-3. doi: 10.1126/science.367.6485.1412. PubMed PMID: 32217705.
3. Hoffmann M, Kleine-Weber H, Schroeder S, Kruger N, Herrler T, Erichsen S, et al. SARS-CoV-2 Cell Entry Depends on ACE2 and TMPRSS2 and Is Blocked by a Clinically Proven Protease Inhibitor. Cell. 2020. doi: 10.1016/j.cell.2020.02.052. PubMed PMID: 32142651; PubMed Central PMCID: PMC7102627.
4. Wan Y, Shang J, Graham R, Baric R S, Li F. Receptor Recognition by the Novel Coronavirus from Wuhan: an Analysis Based on Decade-Long Structural Studies of SARS Coronavirus. Journal of virology. 2020; 94(7). doi: 10.1128/JVI.00127-20. PubMed PMID: 31996437; PubMed Central PMCID: PMC7081895.
5. Walls A C, Xiong X, Park Y J, Tortorici M A, Snijder J, Quispe J, et al. Unexpected Receptor Functional Mimicry Elucidates Activation of Coronavirus Fusion. Cell. 2019; 176(5):1026-39 e15. doi: 10.1016/j.cell.2018.12.028. PubMed PMID: 30712865; PubMed Central PMCID: PMC6751136.
6. Yan R, Zhang Y, Li Y, Xia L, Guo Y, Zhou Q. Structural basis for the recognition of SARS-CoV-2 by full-length human ACE2. Science. 2020; 367(6485):1444-8. doi: 10.126/science.abb2762. PubMed PMID: 32132184.
7. Lan J, Ge J, Yu J, Shan S, Zhou H, Fan S, et al. Structure of the SARS-CoV-2 spike receptor-binding domain bound to the ACE2 receptor. Nature. 2020. doi: 10.1038/s41586-020-2180-5. PubMed PMID: 32225176.
8. Wang C. Li W, Drabek D, Okba N M A, van Haperen R, Osterhaus A, et al. A human monoclonal antibody blocking SARS-CoV-2 infection. Nature communications. 2020; 11(1):2251. doi: 10.1038/s41467-020-16256-y. PubMed PMID: 32366817; PubMed Central PMCID: PMC7198537.
9. Pinto D, Park Y J, Beltramello M, Walls A C, Tortorici M A, Bianchi S, et al. Cross-neutralization of SARS-CoV-2 by a human monoclonal SARS-CoV antibody. Nature, 2020, doi: 10.1038/s41586-020-2349-y. PubMed PMID: 32422645.
10. Zhou J, Rossi J. Aptamers as targeted therapeutics: current potential and challenges. Nature reviews Drug discovery. 2017; 16(3):181-202. doi: 10.1038/nrd.2016.199. PubMed PMID: 27807347.
11. Tuerk C, Gold L. Systematic evolution of ligands by exponential enrichment: RNA ligands to bacteriophage T4 DNA polymerase. Science. 1990; 249(4968):505-10. PubMed PMID: 2200121.
12. Zhou J, Rossi J J. Cell-type-specific, Aptamer-functionalized Agents for Targeted Disease Therapy. Molecular therapy Nucleic acids. 2014; 3:e169. doi: 10.1038/mtna.2014.21. PubMed PMID: 24936916; PubMed Central PMCID: PMC4078761.
13. Sungnak W, Huang N, Becavin C, Berg M, Queen R, Litvinukova M, et al. SARS-CoV-2 entry factors are highly expressed in nasal epithelial cells together with innate immune genes. Nature medicine. 2020; 26(5):681-7. doi: 10.1038/s41591-020-0868-6. PubMed PMID: 32327758.
14. Justin Stebbing A P, Ivan Griffin, Catherine Tucker, Olly Oechsle, Dan Smith, Peter Richardson. COVID-19: combining antiviral and anti-inflammatory treatments. The Lancet Infectious diseases. 2020 doi: https://doi.org/10.1016/S1473-3099(20)30132-8.
15. Zhou P, Yang X L, Wang X G, Hu B, Zhang L, Zhang W, et al. A pneumonia outbreak associated with a new coronavirus of probable bat origin. Nature. 2020; 579(7798):270-3. doi: 10.1038/s41586-020-2012-7. PubMed PMID: 32015507; PubMed Central PMCID: PMC7095418.
16. Patil J S, Sarasija S. Pulmonary drug delivery strategies: A concise, systematic review. Lung India: official organ of Indian Chest Society. 2012; 29(1):44-9. doi: 10.4103/0970-2113.92361. PubMed PMID: 22345913; PubMed Central PMCID: PMC3276033.
17. Li B J, Tang Q, Cheng D, Qin C, Xie F Y, Wei Q, et al. Using siRNA in prophylactic and therapeutic regimens against SARS coronavirus in Rhesus macaque. Nature medicine. 2005; 11(9):944-51. doi: 10.1038/nm1280. PubMed PMID: 16116432; PubMed Central PMCID: PMC7095788.
18. Karras J G, Crosby J R. Guha M, Tung D, Miller D A, Gaarde W A, et al. Anti-inflammatory activity of inhaled IL-4 receptor-alpha antisense oligonucleotide in mice. American journal of respiratory cell and molecular biology. 2007; 36(3):276-85. doi: 10.1165/rcmb.2005-04560C. PubMed PMID: 16990616.
19. Duan W, Chan J H, McKay K, Crosby J R, Choo H H, Leung B P, et al. Inhaled p38alpha mitogen-activated protein kinase antisense oligonucleotide attenuates asthma in mice. American journal of respiratory and critical care medicine. 2005; 171(6):571-8. doi: 10.1164/rccm.200408-10060C. PubMed PMID: 15557129.
20. Crosby J R, Zhao C, Jiang C, Bai D, Katz M, Greenlee S, et al. Inhaled ENaC antisense oligonucleotide ameliorates cystic fibrosis-like lung disease in mice. Journal of cystic fibrosis: official journal of the European Cystic Fibrosis Society. 2017; 16(6):671-80. doi: 10.1016/j.jcf.2017.05.003. PubMed PMID: 28539224.
21. Crosby J R, Guha M, Tung D, Miller D A, Bender B, Condon T P, et al. Inhaled CD86 antisense oligonucleotide suppresses pulmonary inflammation and airway hyper-responsiveness in allergic mice. The Journal of pharmacology and experimental therapeutics. 2007; 321(3):938-46. doi: 10.1124/jpet.106.119214. PubMed PMID: 17389243,
22. Fey R A, Templin M V, McDonald J D, Yu R Z, Hutt J A, Gigliotti A P, et al. Local and systemic tolerability of a 2′O-methoxyethyl antisense oligonucleotide targeting interleukin-4 receptor-alpha delivery by inhalation in mouse and monkey. Inhalation toxicology. 2014; 26(8):452-63. doi: 10.3109/08958378.2014.907587. PubMed PMID: 24932560.
23. Zhou J, Lazar D, Li H, Xia X, Satheesan S, Charlins P, et al. Receptor-targeted aptamer-siRNA conjugate-directed transcriptional regulation of HIV-1. Theranostics. 2018; 8(6):1575-90. doi: 10.7150/thno.23085. PubMed PMID: 29556342; PubMed Central PMCID: PMC5858168.
24. Zhou J, Neff C P, Swiderski P, Li H, Smith D D, Aboellail T, et al. Functional In Vivo Delivery of Multiplexed Anti-HIV-1 siRNAs via a Chemically Synthesized Aptamer With a Sticky Bridge. Molecular therapy: the journal of the American Society of Gene Therapy. 2013; 21(1):192-200. Epub 2012/11/21. doi: mt2012226 [pii]10.1038/mt.2012.226. PubMed PMID: 23164935; PubMed Central PMCID: PMC3538316.
25. Neff C P. Zhou J, Remling L, Kuruvilla J, Zhang J, Li H, et al. An aptamer-siRNA chimera suppresses HIV-1 viral loads and protects from helper CD4(+) T cell decline in humanized mice. Science translational medicine. 2011; 3(66):66ra6. Epub 2011/01/21. doi: 10.1126/scitranslmed.3001581. PubMed PMID: 21248316; PubMed Central PMCID: PMC3138523.
26. Zhou J, Swiderski P, Li H, Zhang J, Neff C P, Akkina R, et al. Selection, characterization and application of new RNA HIV gp 120 aptamers for facile delivery of Dicer substrate siRNAs into HIV infected cells. Nucleic acids research. 2009; 37(9):3094-109. Epub 2009/03/24. doi: 10.1093/nar/gkp185. PubMed PMID: 19304999; PubMed Central PMCID: PMC2685106.
27. Zhou J, Li H, Li S, Zaia J, Rossi J J. Novel dual inhibitory function aptamer-siRNA delivery system for HIV-1 therapy. Molecular therapy: the journal of the American Society of Gene Therapy. 2008; 16(8):1481-9. Epub 2008/05/08. doi: 10.1038/mt.2008.92. PubMed PMID: 18461053; PubMed Central PMCID: PMC2727148.
28. Zhou J, Satheesan S, Li H, Weinberg M S, Morris K V, Burnett J C, et al. Cell-specific RNA aptamer against human CCR5 specifically targets HIV-1 susceptible cells and inhibits HIV-1 infectivity. Chemistry & biology. 2015; 22(3):379-90. doi: 10.1016/j.chembiol.2015.01.005. PubMed PMID: 25754473; PubMed Central PMCID: PMC4369413.
29. Bastings M M C, Helms B A, van Baal I, Hackeng T M, Merkx M, Meijer E W. From phage display to dendrimer display: Insights into multivalent binding. Journal of the American Chemical Society. 2011; 133(17):6636-41.
30. Musumeci D, Montesarchio D. Polyvalent nucleic acid aptamers and modulation of their activity: a focus on the thrombin binding aptamer. Pharmacol Ther. 2012; 136(2):202-15. doi: 10.1016/j.pharmthera.2012.07.011. PubMed PMID: 22850531.
31. McNamara J O, Kolonias D, Pastor F, Mittler R S, Chen L, Giangrande P H, et al. Multivalent 4-1BB binding aptamers costimulate CD8+ T cells and inhibit tumor growth in mice. The Journal of clinical investigation. 2008; 118(1):376-86. PubMed PMID: 18060045.
32. Takahashi M, Wu X, Ho M, Chomchan P, Rossi J J, Burnett J C, et al. High throughput sequencing analysis of RNA libraries reveals the influences of initial library and PCR methods on SELEX efficiency. Scientific reports. 2016; 6:33697. doi: 10.1038/srep33697. PubMed PMID: 27652575; PubMed Central PMCID: PMC5031971.
33. Zhou J, Rossi J J. Evolution of Cell-Type-Specific RNA Aptamers Via Live Cell-Based SELEX. Methods in molecular biology. 2016; 1421:191-214. doi: 10.1007/978-1-4939-3591-8_16. PubMed PMID: 26965267.
34. Zhou J, Tiemann K, Chomchan P, Alluin J, Swiderski P, Burnett J, et al. Dual functional BAFF receptor aptamers inhibit ligand-induced proliferation and deliver siRNAs to NHL cells. Nucleic acids research. 2013; 41(7):4266-83. doi: 10.1093/nar/gkt125. PubMed PMID: 23470998; PubMed Central PMCID: PMC3627597.
35. Linlin Bao W D, Baoying Huang, Hong Gao, Jiangning Liu, Lili Ren, Qiang Wei, Pin Yu, Yanfeng Xu, Feifei Qi, Yajin Qu, Fengdi Li. Qi Lv, Wenling Wang, Jing Xue, Shuran Gong, Mingya Liu, Guanpeng Wang, Shunyi Wang, Zhiqi Song, Linna Zhao, Peipei Liu, Li Zhao, Fei Ye, Huijuan Wang, Weimin Zhou, Na Zhu, Wei Zhen, Haisheng Yu, Xiaojuan Zhang, Li Guo, Lan Chen, Conghui Wang, Ying Wang, Xinming Wang, Yan Xiao, Qiangming Sun, Hongqi Liu, Fanli Zhu, Chunxia Ma, Lingmei Yan, Mengli Yang, Jun Han, Wenbo Xu, Wenjie Tan, Xiaozhong Peng, Qi Jin, Guizhen Wu, Chuan Qin. The Pathogenicity of SARS-CoV-2 in hACE2 Transgenic Mice. BioRxiV. 2020. Epub Feb. 28, 2020. doi: https://doi.org/10.1101/2020.02.07.939389.
36. McCray P B, Jr., Pewe L, Wohlford-Lenane C, Hickey M, Manzel L, Shi L, et al. Lethal infection of K18-hACE2 mice infected with severe acute respiratory syndrome coronavirus. Journal of virology. 2007; 81(2):813-21. doi: 10.1128/JVI.02012-06. PubMed PMID: 17079315; PubMed Central PMCID: PMC1797474.
37. B Korber W F, S Gnanakaran, H Yoon, J Theiler, W Abfalterer, B Foley, EE Giorgi, T Bhattacharya, MD Parker, DG Partridge, CM Evans, TI de Silva, on behalf of the Sheffield COVID-19 Genomics Group, CC LaBranche, DC Montefiori. Spike mutation pipeline reveals the emergence of a more transmissible form of SARS-CoV-2. BioRxiV 2020. doi: https://doi.org/10.1101/2020.04.29.069054.

1-50. (canceled)

51. A nucleic acid molecule comprising a nucleic acid sequence, wherein the nucleic acid sequence is at least 85% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO: 11.

52. The nucleic acid molecule of claim 51, wherein the nucleic acid sequence is at least 95% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, or SEQ ID NO: 11.

53. A nucleic acid molecule comprising a nucleic acid sequence, wherein the nucleic acid sequence comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:14, SEQ ID NO:15, SEQ ID NO:16, or SEQ ID NO:17.

54. The nucleic acid molecule of claim 51, wherein the nucleic acid sequence is an RNA sequence.

55. The nucleic acid molecule of claim 54 wherein the RNA sequence further comprises a nucleotide sequence attached at the 5′ end; a nucleotide sequence attached at the 3′ end; or a nucleotide sequence attached at the 5′ end and a nucleotide sequence attached at the 3′ end.

56. The nucleic acid molecule of claim 54, wherein the RNA sequence further comprises a nucleotide sequence attached at the 5′ end and a nucleotide sequence attached at the 3′ end, wherein:
(a) the nucleotide sequence attached at the 5′ end comprises a stem loop structure and the nucleotide sequence attached at the 3′ end comprises a stem loop structure;
(b) the nucleotide sequence attached at the 5′ end comprises a stem loop structure or the nucleotide sequence attached at the 3′ end comprises a stem loop structure; or
(c) the nucleotide sequence attached at the 5′ end and the nucleotide sequence attached at the 3′ end are combined to form a stem loop structure.

57. The nucleic acid molecule of claim 54, wherein the RNA sequence further comprises a nucleotide sequence attached at the 5′ end and a nucleotide sequence attached at the 3′ end; and wherein the nucleic acid molecule comprises a stem loop structure comprising a portion of the RNA sequence, a portion of the nucleotide sequence attached to the 5′ end, and a portion of the nucleotide sequence attached to the 3′ end.

58. The nucleic acid molecule of claim 54, wherein the RNA sequence comprises one or more stem loop structures.

59. The nucleic acid molecule of claim 55, wherein the nucleotide sequence attached to the 5′ end is at least 90% identical to SEQ ID NO:12.

60. The nucleic acid molecule of claim 55, wherein the nucleotide sequence attached to the 5′ end comprises SEQ ID NO:12.

61. The nucleic acid molecule of claim 55, wherein the nucleotide sequence attached to the 3′ end is at least 90% identical to SEQ ID NO:13.

62. The nucleic acid molecule of claim 55, wherein the nucleotide sequence attached to the 3′ end comprises SEQ ID NO:13.

63. The nucleic acid molecule of claim 51, wherein the nucleic acid molecule is RNase-resistant.

64. A pharmaceutical composition comprising a therapeutically effective amount of the nucleic acid molecule of claim 51 and one or more additives or excipients.

65. The pharmaceutical composition of claim 64, further comprising a vaccine, an anti-inflammatory agent, an antibody, a short-acting beta agonist, an antipyretic agent or an immune booster.

66. A method of treating or preventing a respiratory tract infection in a subject in need thereof, the method comprising administering to the subject a therapeutically effective amount of the nucleic acid molecule of claim 54.

67. The method of claim 66, wherein the respiratory tract infection is in the upper respiratory tract of the subject or in the lower respiratory tract of the subject.

68. The method of claim 66, wherein the respiratory tract infection is a severe acute respiratory syndrome infection.

69. A method of treating or preventing COVID-19 in a subject in need thereof, the method comprising administering to the subject a therapeutically effective amount of the nucleic acid molecule of claim 54.

70. A method of treating or preventing a respiratory tract infection in a subject in need thereof, the method comprising administering to the subject a therapeutically effective amount of the nucleic acid molecule of claim 56.
